(19) United States
(12) Patent Application Publication
(10) Pub. No.: US 2017/0174633 A1
(43) Pub. Date: Jun. 22, 2017

(54) PYRAZOLE COMPOUNDS SELECTIVE FOR NEUROTENSIN 2 RECEPTOR

(71) Applicant: RESEARCH TRIANGLE INSTITUTE, Research Triangle Park, NC (US)

(72) Inventors: James B. THOMAS, Efland, NC (US); Brian P. GILMOUR, Mebane, NC (US); Angela M. GIDDINGS, Carrboro, NC (US); Robert W. WIETHE, Durham, NC (US); Keith R. WARNER, Raleigh, NC (US); Srinivas OLEPU, Morrisville, NC (US)

(21) Appl. No.: 15/128,725

(22) PCT Filed: Mar. 24, 2015
(86) PCT No.: PCT/US2015/022179

§ 371 Date: Sep. 23, 2016

Related U.S. Application Data
(60) Provisional application No. 61/970,023, filed on Mar. 25, 2014.

(51) Int. Cl. C07D 231/22; A61K 31/415

(52) U.S. Cl. CPC C07D 231/22; A61K 31/415

(57) ABSTRACT

This invention relates generally to the discovery of pyrazole compounds selective for the neurotensin receptor 2 (NTR2) and uses thereof.

### CROSS REFERENCE TO RELATED APPLICATIONS

This application claims the benefit of U.S. Provisional Appn. 61/970,023 filed Mar. 25, 2014, Thomas et al., entitled “PYRAZOLE COMPOUNDS SELECTIVE FOR NEUROTENSIN 2 RECEPTOR”, attorney reference no. 121/38 PROV which is hereby incorporated by reference in its entirety.

### STATEMENT REGARDING FEDERALLY SPONSORED RESEARCH OR DEVELOPMENT

This invention was made with government support under Grant No. DA029961 awarded by the National Institutes on Drug Abuse. The United States Government has certain rights in the invention.

### 1. FIELD OF THE INVENTION

This invention relates generally to the discovery of pyrazole compounds selective for the neurotensin receptor 2 (NTS2) and uses thereof.

### 2. BACKGROUND OF THE INVENTION

### 2.1. Introduction

Neurotensin (NT) is a tridecapeptide (Glu-Leu-Tyr-Glu-Asn-Lys-Pro-Arg-Arg-Pro-Tyr-Ile-Leu) that was identified forty years ago from bovine hypothalamus¹. NT functions as a neurotransmitter and neuromodulator demonstrating a range of biological actions. In the CNS, NT is co-localized with and regulates the action of mesolimbic and nigrastriatal dopamine^{2,6} as well as mediating non-opioid analgesia and hypothermia^{7,8}. It is believed that NT operates primarily through interaction with two G-protein coupled receptors NTS1 and NTS2 (also referred to as NTR1, NTR2, NTRH, NTRL and others) to regulate these activities though a third receptor, NTS3, is known to exist bearing but a single transmembrane domain⁹⁻¹³. The ability of the NT receptor system to regulate CNS dopamine led researchers to postulate that NT might be an endogenous neuroleptic¹⁴ and that drugs acting via the NT system might therefore be useful as anti-psychotic agents^{15,16}.

Abuse of methamphetamine also produces profound disruption of dopamine flow in the mesolimbic and nigrastriatal dopamine networks with chronic methamphetamine exposure eliciting behaviors resembling schizophrenia^{17, 18}. It is thus no surprise that compounds acting via the NT network are now being investigated as potential treatments for methamphetamine abuse^{19, 20}.

The neurotensin system also plays an important role in the pain processing network^{7,21}. The non-opioid analgesia mediated by the NT system has also generated much interest over the years as a potential means of circumventing the side effects produced by opioid analgesics including addiction and respiratory depression. NT mediated analgesia has been demonstrated with compounds selective for both the NTS1 and NTS2 receptors as well as non-selective compounds²²⁻²⁷. Beyond this, there is now substantial evidence that both NTS1 and NTS2 can mediate relief from chronic or neuropathic pain, a persistent form of pain that arises from nerve damage²⁸. This type of pain is difficult to treat with current drugs and does not respond well to opioid therapy²⁹. Taken together with their neuroleptic activity, it is easy to understand why the development of compounds acting via the NT network has engendered so much interest.

### 3. SUMMARY OF THE INVENTION

In particular non-limiting embodiments, the present invention provides a compound represented by the Formula I:

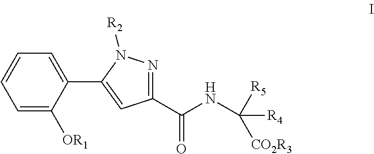

or a pharmaceutically acceptable salt, a prodrug, or a salt of a prodrug, wherein R₁ is adamantanyl, aryl, C₁₋₈ alkyl, C₁₋₈ alkyl(aryl), C₁₋₈ alkyl (C₃₋₈ cycloalkyl), C₂₋₈ alkenyl, C₃₋₈ alkynyl, C₃₋₈ cycloalkyl; R₂ is aryl, C₁₋₈ alkyl(aryl); R₃ is adamantanyl, aryl, C₁₋₈ alkyl, C₁₋₈ alkyl(aryl), C₁₋₈ alkyl (C₃₋₈ cycloalkyl), C₂₋₈ alkenyl, C₃₋₈ alkynyl, C₃₋₈ cycloalkyl or H; and R₄ and R₅ are independently adamantanyl, aryl, C₁₋₈ alkyl, C₁₋₈ alkyl(aryl), C₁₋₈ alkyl (C₃₋₈ cycloalkyl), C₂₋₈ alkenyl, C₃₋₈ alkynyl, C₃₋₈ cycloalkyl, or H; or R₄ and R₅ together make a 4-8 member ring which may be substituted with one or more heteroatoms.

R₁ may be C₁₋₈ alkyl or C₁₋₃ alkyl. R₂ may be aryl and the aryl moiety may be substituted with a halogen. In specific embodiments, R₂ may be fluoroaryl, fluorophenyl, chloroaryl, chloroquinolinyl, an unsubstituted aryl, or napthyl.

R₄ and R₅ may together make a 4-8 member ring which may be a C₅₋₈ cycloalkyl ring. R₁ may be C₁₋₃ alkyl and R₂ may be fluoroaryl.

The compound may have the Formula of any of compounds 7b, 14b, 15b, 16b, 17b, 18b, 19b, 20b, 21b, 22b, 23b, 24b, 25b, 26b, 27b, 28b, 29b, 30 or 31 as set forth in Table 2 or 3.

A pharmaceutical composition comprising at least one pharmaceutically acceptable excipient and a therapeutically effective amount of the compound of Formula I. The pharmaceutical composition may have the compound present in amount effective for the treatment of pain which may be chronic pain or neuropathic pain.

Also provided is a method of treating a neurotensin 2 receptor (NTS2)-related disorder in a subject which comprises administering to the subject the compound of claim 1. The neurotensin 2 receptor (NTS2)-related disorder may be pain such as chronic pain or neuropathic pain.

Furthermore, the compounds described herein may be useful for the treatment of cancers, such as prostate cancer. See, Swift, S. L.; Burns, J. E.; Maitland, N. J. Cancer Res 2010, 70, 347-356, the contents of which are hereby incorporated by reference.

### 4. BRIEF DESCRIPTION OF THE FIGURES

FIG. 1 is Chart 1 showing the structure of compounds 1, 2, 3, 4a, 4b, 5a, 5b, 6, and 7b. (1) Arg-Arg-Pro-Tyr-Ile-Leu; (2) Boc-Arg-Arg-Pro-Tyr-y(CH₂NH)-Ile-Leu; (3) (N^{α}Me)Arg-Lys-Pro-D-3,1-Nal-tLeu-Leu; (4a) Arg-Arg-Pro-N-homo-Tyr-Ile-Leu; and (4b) (N^{α}Me)Arg-Lys-Pro-N-homo-Tyr-Ile-Leu.

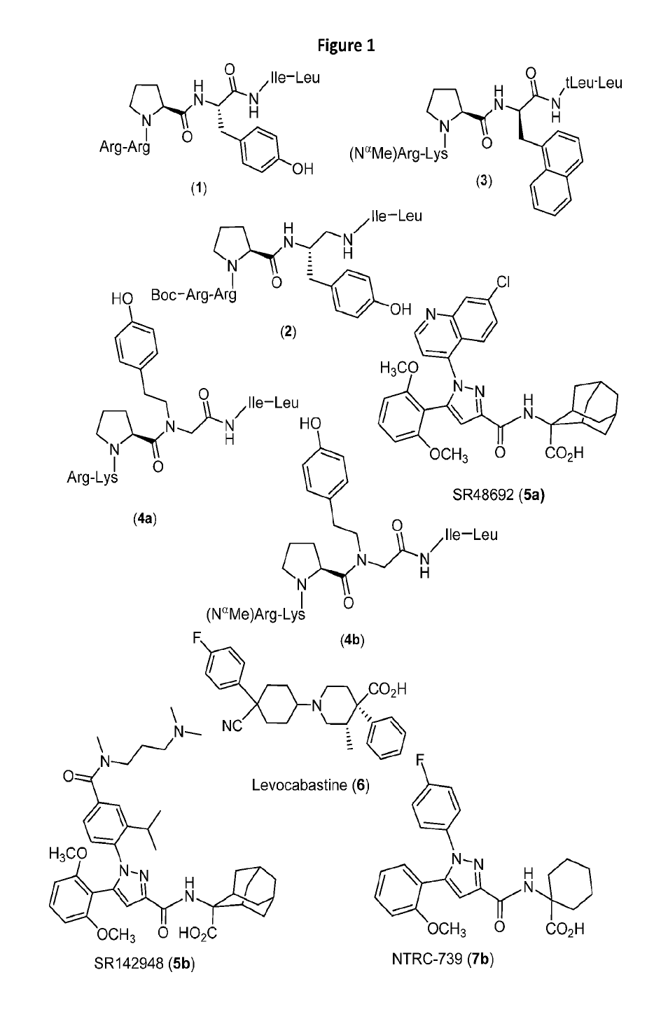

FIG. 2 is Chart 2 showing the amines, amino acids and amino acid esters used to prepare compounds 7b, 13, 14b-29b, 30 and 31.

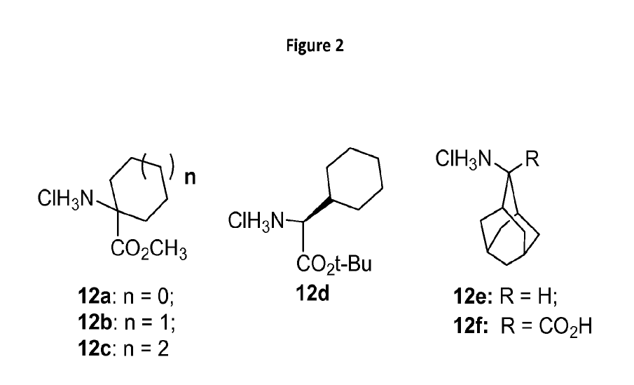

FIG. 3 is Scheme 1 showing the synthesis of key pyrazole intermediates 11a-11j. 8a: X=2,6-diMeO; Y═H; 8b: X=2,6-diMeO; Y=Et; 8c: X=2,5-diMeO; Y═H; 8d: X=2,4-diMeO; Y═H; 8e: X=2,6-diF; Y═H; and 8f: X=2-MeO; Y═H. 10a, 11a: X=2,6-diMeO; Y═H; Z=7-Cl-quinolin-4-yl; 10b, 11b: X=2,6-diMeO; Y=Et; Z=7-Cl-quinolin-4-yl; 10c, 11c: X=2,5-diMeO; Y═H; Z=7-Cl-quinolin-4-yl; 10d, 11d: X=2,4-diMeO; Y═H; Z=7-Cl-quinolin-4-yl; 10e, 11e: X=2,6-diF; Y═H; Z=7-Cl-quinolin-4-yl; 10f, 11f: X=2-MeO; Y═H; Z=7-Cl-quinolin-4-yl; 10g, 11g: X=2,6-diMeO; Y═H; Z=1-napthyl; 10h, 11h: X=2-MeO; Y═H; Z=1-napthyl; 10i, 11i: X=2,6-diMeO; Y═H; Z=4-F-phenyl; and 10j, 11j: X=2-MeO; Y═H; Z=4-F-phenyl.

Reagents and conditions: (i) HOAc, HCl, and 9a (7-chloroquinolin-4-yl)hydrazine.HCl) or 9b (1-napthylhydrazine.HCl) or 9c (4-fluorophenylhydrazine.HCl), reflux 4 h; (ii) LiOH 3 eq, dioxane, RT 16h.

FIG. 4 is Scheme 2 showing the synthesis of target compounds 7b, 13, 14a-29b, 30 and 31. 13: X=2,6-diMeO, R═H; Z=7-Cl-quinolin-4-yl and 30: X=2,6-diMeO, R═CO2H; Z=4-F-phenyl and 31: X=2-MeO, R═CO2H; Z=4-F-phenyl. 7a, 20-23a, 26a, 27a, 29a: R=Me. 7b, 20-23b, 26b, 27b, 29b: R═H. 7a,b X=2-MeO; Z=4-F-phenyl; n=1; 20a,b: X=2,6-diMeO; Z=7-Cl-quinolin-4-yl; n=2; 21a,b: X=2,6-diMeO; Z=7-Cl-quinolin-4-yl; n=1; 22a,b: X=2,6-diMeO; Z=7-Cl-quinolin-4-yl; n=0; 23a,b: X=2-MeO; Z=7-Cl-quinolin-4-yl; n=1; 26a,b: X=2,6-diMeO; Z=1-napthyl; n=1; 27a,b: X=2-MeO; Z=1-napthyl; n=1; and 29a,b: X=2,6-diMeO; Z=4-F-phenyl; n=1.

14a-19a, 24a, 25a, 28a: R=t-Bu. 14b-19b, 24b, 25b, 28b: R═H. 14a,b: X=2,6-diMeO; Y═H; Z=7-Cl-quinolin-4-yl; 15a,b: X=2,5-diMeO; Y═H; Z=7-Cl-quinolin-4-yl; 16a,b: X=2,4-diMeO; Y═H; Z=7-Cl-quinolin-4-yl; 17a,b: X=2-MeO; Y═H; Z=7-Cl-quinolin-4-yl; 18a,b: X=2,6-diF; Y═H; Z=7-Cl-quinolin-4-yl; 19a,b: X=2,6-diMeO; Y=Et; Z=7-Cl-quinolin-4-yl; 24a,b: X=2,6-diMeO; Y═H; Z=1-napthyl; 25a,b: X=2-MeO; Y═H; Z=1-napthyl; 28a,b: X=2,6-diMeO; Y═H; Z=4-F-phenyl.

Reagents and conditions: (i) HBTU, Et₃N, CH₂Cl₂, 2-aminoadamantane.HCl (12e); (ii) SOCl₂, toluene; (iii) NaOH, THF, 12f; (iv) HBTU, Et₃N, CH₂Cl₂, amino acid ester 12d; (v) TFA, CH₂Cl₂; (vi) HBTU, Et₃N, CH₂Cl₂, amino acid ester 12a-c; (vii) LiOH, dioxane.

### 5. DETAILED DESCRIPTION OF THE INVENTION

This invention provides compounds acting at the neurotensin 2 receptor (NTS2) that are active in animal models of chronic pain and those selective for NTS2 versus the neurotensin 1 receptor (NTS1) that do not display the side effects of hypotension and hypothermia. Levocabastine (6, Livostin™), a non-peptide H1 histamine antagonist, is also active at NTS2 and was found to be selective for NTS2 versus NTS1 many years ago. Recently this compound has been shown to be efficacious in a model of neuropathic pain. With the aim of identifying novel compounds selective for NTS2, the literature reports of calcium release stimulated by the potent NTS1 antagonists SR48692 (5a, Meclinertant™) and SR142948 (5b) in CHO cells stably expressing the NTS2 receptor, prompted us to follow up these findings to determine if this calcium response could be used to guide structure activity relationship (SAR) studies. In testing, we found levocabastine to be a potent partial agonist of calcium release in this assay relative to 5b. Using these NTS2 cells in a FLIPR assay in concert with an NTS1 calcium release assay, we were able to identify a levocabastine—like NTS2 selective compound 1-({[1-(4-fluorophenyl)-5-(2-methoxyphenyl)-1H-pyrazol-3-yl]carbonyl}amino)cyclohexane carboxylic acid (NTRC-739, 7b) starting from the non-selective compound SR48692 (5a). Radioligand binding experiments carried out on the test compounds described herein confirmed a positive correlation between binding affinity at NTS2 and NTS2 mediated calcium mobilization. Comparison of the data obtained for 7b from NTS2 and NTS1 binding assays provided additional confirmation of the selectivity of compound 7b for NTS2.

### 5.1. Definitions

“Alkenyl” refers to an unsaturated branched, straight-chain or cyclic alkyl group having at least one carbon-carbon double bond derived by the removal of one hydrogen atom from a single carbon atom of a parent alkene. The group may be in either the Z- and E-forms (or cis or trans conformation) about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl; and the like. The alkenyl group may be substituted or unsubstituted. In certain embodiments, an alkenyl group has from 2 to 20 carbon atoms and in other embodiments from 2 to 8 carbon atoms.

“Alkoxy” refers to a radical —OR where R represents an alkyl, alkyl, cycloalkyl, aryl, or heteroaryl group as defined herein. Representative examples include, but are not limited to, methoxy, ethoxy, propoxy, butoxy, cyclohexyloxy, and the like.

“Alkyl” refers to a saturated, branched or straight-chain monovalent hydrocarbon group derived by the removal of one hydrogen atom from a single carbon atom of a parent alkane. Typical alkyl groups include, but are not limited to, methyl, ethyl, propyls such as propan-1-yl, propan-2-yl, and cyclopropan-1-yl, butyls such as butan-1-yl, butan-2-yl, 2-methyl-propan-1-yl, 2-methyl-propan-2-yl, cyclobutan-1-yl, tert-butyl, and the like. The alkyl group may be substituted or unsubstituted. In certain embodiments, an alkyl group comprises from 1 to 20 carbon atoms. Alternatively, an alkyl group may comprise from 1 to 8 carbon atoms.

“Alkyl(aryl)” refers to an acyclic alkyl group in which one of the hydrogen atoms bonded to a carbon atom, typically a terminal or sp³ carbon atom, is replaced with an aryl group. Typical alkyl(aryl) groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. In certain embodiments, an alkyl(aryl) group can be (C₆₋₂₀) alkyl(aryl) e.g., the alkyl group may be (C₁₋₁₀) and the aryl moiety may be (C₅₋₁₀).

“Alkynyl” refers to an unsaturated branched or straight-chain having at least one carbon-carbon triple bond derived by the removal of one hydrogen atom from a single carbon atom of a parent alkyne. Typical alkynyl groups include, but are not limited to, ethynyl, propynyl, butenyl, 2-pentynyl, 3-pentynyl, 2-hexynyl, 3-hexynyl and the like. The alkynyl group may be substituted or unsubstituted. In certain embodiments, an alkynyl group has from 3 to 20 carbon atoms and in other embodiments from 3 to 8 carbon atoms.

“Aryl” refers to a monovalent aromatic hydrocarbon group derived by the removal of one hydrogen atom from a single carbon atom of a parent aromatic ring system. Aryl encompasses 5- and 6-membered carbocyclic aromatic rings, for example, benzene or cyclopentadiene; bicyclic ring systems wherein at least one ring is carbocyclic and aromatic, for example, naphthalene, indane; or two aromatic ring systems, for example benzyl phenyl, biphenyl, diphenylethane, diphenylmethane. The aryl group may be substituted or unsubstituted.

“Cycloalkyl” refers to a saturated or unsaturated cyclic alkyl group. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane, and the like. The cycloalkyl group may be substituted or unsubstituted. In certain embodiments, the cycloalkyl group can be C₃₋₁₀ cycloalkyl, such as, for example, C₆ cycloalkyl.

“Disease” refers to any disease, disorder, condition, symptom, or indication.

“Halogen” refers to a fluoro, chloro, bromo, or iodo group.

“Heteroaryl” refers to a monovalent heteroaromatic group derived by the removal of one hydrogen atom from a single atom of a parent heteroaromatic ring system. Heteroaryl encompasses: 5- to 7-membered aromatic, monocyclic rings containing one or more, for example, from 1 to 4, or in certain embodiments, from 1 to 3, heteroatoms chosen from N, O, and S, with the remaining ring atoms being carbon; and polycyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring. The heteroaryl group may be substituted or unsubstituted.

For example, heteroaryl includes a 5- to 7-membered heteroaromatic ring fused to a 5- to 7-membered cycloalkyl ring and a 5- to 7-membered heteroaromatic ring fused to a 5- to 7-membered heterocycloalkyl ring. For such fused, bicyclic heteroaryl ring systems wherein only one of the rings contains one or more heteroatoms, the point of attachment may be at the heteroaromatic ring or the cycloalkyl ring. When the total number of S and O atoms in the heteroaryl group exceeds 1, those heteroatoms are not adjacent to one another. In certain embodiments, the total number of S and O atoms in the heteroaryl group is not more than 2. In certain embodiments, the total number of S and O atoms in the aromatic heterocycle is not more than 1. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, the heteroaryl group can be between 5 to 20 membered heteroaryl, such as, for example, a 5 to 10 membered heteroaryl. In certain embodiments, heteroaryl groups can be those derived from thiophene, pyrrole, benzothiophene, benzofuran, indole, pyridine, quinoline, imidazole, oxazole, and pyrazine.

“Pharmaceutically acceptable” refers to generally recognized for use in animals, and more particularly in humans.

“Pharmaceutically acceptable salt” refers to a salt of a compound that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, dicyclohexylamine, and the like.

“Pharmaceutically acceptable excipient,” “pharmaceutically acceptable carrier,” or “pharmaceutically acceptable adjuvant” refer, respectively, to an excipient, carrier or adjuvant with which at least one compound of the present disclosure is administered. “Pharmaceutically acceptable vehicle” refers to any of a diluent, adjuvant, excipient or carrier with which at least one compound of the present disclosure is administered.

“Stereoisomer” refers to an isomer that differs in the arrangement of the constituent atoms in space. Stereoisomers that are mirror images of each other and optically active are termed “enantiomers,” and stereoisomers that are not mirror images of one another and are optically active are termed “diastereoisomers.”

“Subject” includes mammals and humans. The terms “human” and “subject” are used interchangeably herein.

“Substituted” refers to a group in which one or more hydrogen atoms are each independently replaced with the same or different substituent(s). Typical substituents include, but are not limited to, CO₂H, halogen, hydroxyl, —N₃, —NH₂, —SO₍₁₋₃₎H, or —SH.

“Therapeutically effective amount” refers to the amount of a compound that, when administered to a subject for treating a disease, or at least one of the clinical symptoms of a disease or disorder, is sufficient to affect such treatment for the disease, disorder, or symptom. The “therapeutically effective amount” can vary depending on the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be readily apparent to those skilled in the art or capable of determination by routine experimentation.

“Treating” or “treatment” of any disease or disorder refers to arresting or ameliorating a disease, disorder, or at least one of the clinical symptoms of a disease or disorder, reducing the risk of acquiring a disease, disorder, or at least one of the clinical symptoms of a disease or disorder, reducing the development of a disease, disorder or at least one of the clinical symptoms of the disease or disorder, or reducing the risk of developing a disease or disorder or at least one of the clinical symptoms of a disease or disorder. “Treating” or “treatment” also refers to inhibiting the disease or disorder, either physically, (e.g., stabilization of a discernible symptom), physiologically, (e.g., stabilization of a physical parameter), or both, or inhibiting at least one physical parameter which may not be discernible to the subject. Further, “treating” or “treatment” refers to delaying the onset of the disease or disorder or at least symptoms thereof in a subject which may be exposed to or predisposed to a disease or disorder even though that subject does not yet experience or display symptoms of the disease or disorder.

### 5.2. Pharmaceutically Acceptable Compositions

Provided herein are pharmaceutical compositions comprising a selective NTS2 compound as an active ingredient, or a pharmaceutically acceptable salt, solvate or hydrate thereof in combination with a pharmaceutically acceptable vehicle, carrier, diluent, or excipient, or a mixture thereof.

The compound provided herein may be administered alone, or in combination with one or more other compounds provided herein. The pharmaceutical compositions that comprise a selective NTS2 compound can be formulated in various dosage forms for oral, parenteral, and topical administration. The pharmaceutical compositions can also be formulated as modified release dosage forms, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see, Remington: The Science and Practice of Pharmacy, 21st Ed., Lippincott, Williams & Wilkins, Baltimore, Md., 2006; Modified-Release Drug Delivery Technology, Rathbone et al., Eds., Drugs and the Pharmaceutical Science, Marcel Dekker, Inc.: New York, N.Y., 2003; Vol. 126).

In one embodiment, the pharmaceutical compositions are provided in a dosage form for oral administration, which comprise a compound provided herein, e.g., a selective NTS2 compound or a pharmaceutically acceptable salt, solvate or hydrate thereof; and one or more pharmaceutically acceptable excipients or carriers.

In another embodiment, the pharmaceutical compositions are provided in a dosage form for parenteral administration, which comprise a selective NTS2 compound or a pharmaceutically acceptable salt, solvate or hydrate thereof; and one or more pharmaceutically acceptable excipients or carriers.

In yet another embodiment, the pharmaceutical compositions are provided in a dosage form for topical administration, which comprise a selective NTS2 compound or a pharmaceutically acceptable salt, solvate or hydrate thereof; and one or more pharmaceutically acceptable excipients or carriers.

The pharmaceutical compositions provided herein can be provided in a unit-dosage form or multiple-dosage form. A unit-dosage form, as used herein, refers to physically discrete a unit suitable for administration to a human and animal subject, and packaged individually as is known in the art. Each unit-dose contains a predetermined quantity of an active ingredient(s) sufficient to produce the desired therapeutic effect, in association with the required pharmaceutical carriers or excipients. Examples of a unit-dosage form include an ampoule, syringe, and individually packaged tablet and capsule. A unit-dosage form may be administered in fractions or multiples thereof. A multiple-dosage form is a plurality of identical unit-dosage forms packaged in a single container to be administered in segregated unit-dosage form. Examples of a multiple-dosage form include a vial, bottle of tablets or capsules, or bottle of pints or gallons. The pharmaceutical compositions provided herein can be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In one embodiment, the therapeutically effective dose is from about 0.1 mg to about 2,000 mg per day of a compound provided herein. The pharmaceutical compositions therefore should provide a dosage of from about 0.1 mg to about 2000 mg of the compound. In certain embodiments, pharmaceutical dosage unit forms are prepared to provide from about 1 mg to about 2000 mg, from about 10 mg to about 1000 mg, from about 20 mg to about 500 mg or from about 25 mg to about 250 mg of the essential active ingredient or a combination of essential ingredients per dosage unit form. In certain embodiments, the pharmaceutical dosage unit forms are prepared to provide about 10 mg, 20 mg, 25 mg, 50 mg, 100 mg, 250 mg, 500 mg, 1000 mg or 2000 mg of the essential active ingredient.

5.2.1. Parental Administration

The pharmaceutical compositions provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration.

The pharmaceutical compositions provided herein can be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration can include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents include those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including a-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

The pharmaceutical compositions provided herein can be formulated for single or multiple dosage administration. The single dosage formulations are packaged in an ampoule, a vial, or a syringe. The multiple dosage parenteral formulations must contain an antimicrobial agent at bacteriostatic or fungistatic concentrations. All parenteral formulations must be sterile, as known and practiced in the art.

In one embodiment, the pharmaceutical compositions are provided as ready-to-use sterile solutions. In another embodiment, the pharmaceutical compositions are provided as sterile dry soluble products, including lyophilized powders and hypodermic tablets, to be reconstituted with a vehicle prior to use. In one embodiment, the lyophilized nanoparticles are provided in a vial for reconstitution with a sterile aqueous solution just prior to injection. In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions. The pharmaceutical compositions provided herein can be formulated as immediate or modified release dosage forms, including delayed-, sustained, pulsed-, controlled, targeted-, and programmed-release forms.

The pharmaceutical compositions can be formulated as a suspension, solid, semi-solid, or thixotropic liquid, for administration as an implanted depot.

5.2.2. Oral Administration Compositions

The pharmaceutical compositions provided herein can be provided in solid, semisolid, or liquid dosage forms for oral administration. As used herein, oral administration also includes buccal, lingual, and sublingual administration. Suitable oral dosage forms include, but are not limited to, tablets, fastmelts, chewable tablets, capsules, pills, troches, lozenges, pastilles, cachets, pellets, medicated chewing gum, bulk powders, effervescent or non-effervescent powders or granules, solutions, emulsions, suspensions, wafers, sprinkles, elixirs, and syrups. In addition to the active ingredient(s), the pharmaceutical compositions can contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of fish moss, panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of a disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W. R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation.

The pharmaceutical compositions provided herein can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Hydrophilic polymer formulations have been widely used for improved oral availability such as ethylene oxides, hydroxy propyl methyl cellulose (HPC), poly(ethylene oxide) (PEO), polyvinyl alcohol (PVA), poly(hydroxyethylmethyl acrylate) methyl methacrylate (PHEMA), or vinyl acetate (PCT Pub. No. WO1999/37302 (Alvarez et al.); Dimitrov & Lambov, 1999, Int J Pharm 189 105-111; Zhang et al., 1990, Proc Int. Symp Controlled Release Bioact. Mater. 17, 333, the contents of which are hereby incorporated by reference in their entirety). Film coating imparts the same general characteristics as sugar coating. Multiple compressed tablets are compressed tablets made by more than one compression cycle, including layered tablets, and press-coated or dry-coated tablets.

The tablet dosage forms can be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545, the contents of which are hereby incorporated by reference in their entirety. The capsules may also be coated as known by those of skill in the art in order to modify or sustain dissolution of the active ingredient.

The pharmaceutical compositions provided herein can be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations can further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration can be also provided in the forms of liposomes, micelles, microspheres, or nanosystems. Micellar dosage forms can be prepared as described in U.S. Pat. No. 6,350,458, the content of which is hereby incorporated by reference in its entirety.

The pharmaceutical compositions provided herein can be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms. The pharmaceutical compositions provided herein can be formulated as immediate or modified release dosage forms, including delayed-, sustained, pulsed-, controlled, targeted-, and programmed-release forms.

The pharmaceutical compositions provided herein can be co-formulated with other active ingredients which do not impair the desired therapeutic action, or with substances that supplement the desired action.

5.2.3. Topical Administration

The pharmaceutical compositions provided herein can be administered topically to the skin, orifices, or mucosa. The topical administration, as used herein, includes (intra)dermal, conjunctival, intracorneal, intraocular, ophthalmic, auricular, transdermal, nasal, vaginal, urethral, respiratory, and rectal administration.

The pharmaceutical compositions provided herein can be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, dermal patches. The topical formulation of the pharmaceutical compositions provided herein can also comprise liposomes, micelles, microspheres, nanosystems, and mixtures thereof

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical compositions can also be administered topically by electroporation, iontophoresis, phonophoresis, sonophoresis, or microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions provided herein can be provided in the forms of ointments, creams, and gels. Suitable ointment vehicles include oleaginous or hydrocarbon vehicles, including lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include crosslinked acrylic acid polymers, such as carbomers, carboxypolyalkylenes, CARBOPOL®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein can be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas. These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions provided herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used. Rectal and vaginal suppositories may be prepared by the compressed method or molding. The typical weight of a rectal and vaginal suppository is about 2 to about 3 g.

The pharmaceutical compositions provided herein can be administered ophthalmically in the forms of solutions, suspensions, ointments, emulsions, gel-forming solutions, powders for solutions, gels, ocular inserts, and implants.

The pharmaceutical compositions provided herein can be administered intranasally or by inhalation to the respiratory tract. The pharmaceutical compositions can be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions can also be provided as a dry powder for insufflation, alone or in combination with an inert carrier such as lactose or phospholipids; and nasal drops. For intranasal use, the powder can comprise a bioadhesive agent, including chitosan or cyclodextrin.

Solutions or suspensions for use in a pressurized container, pump, spray, atomizer, or nebulizer can be formulated to contain ethanol, aqueous ethanol, or a suitable alternative agent for dispersing, solubilizing, or extending release of the active ingredient provided herein, a propellant as solvent; and/or a surfactant, such as sorbitan trioleate, oleic acid, or an oligolactic acid.

The pharmaceutical compositions provided herein can be micronized to a size suitable for delivery by inhalation, such as about 50 micrometers or less, or about 10 micrometers or less. Particles of such sizes can be prepared using a comminuting method known to those skilled in the art, such as spiral jet milling, fluid bed jet milling, supercritical fluid processing to form nanoparticles, high pressure homogenization, or spray drying.

Capsules, blisters and cartridges for use in an inhaler or insufflator can be formulated to contain a powder mix of the pharmaceutical compositions provided herein; a suitable powder base, such as lactose or starch; and a performance modifier, such as 1-leucine, mannitol, or magnesium stearate. The lactose may be anhydrous or in the form of the monohydrate. Other suitable excipients or carriers include dextran, glucose, maltose, sorbitol, xylitol, fructose, sucrose, and trehalose. The pharmaceutical compositions provided herein for inhaled/intranasal administration can further comprise a suitable flavor, such as menthol and levomenthol, or sweeteners, such as saccharin or saccharin sodium.

The pharmaceutical compositions provided herein for topical administration can be formulated to be immediate release or modified release, including delayed-, sustained-, pulsed-, controlled-, targeted, and programmed release.

### 5.3. Modified Release Formulations

The pharmaceutical compositions provided herein can be formulated as a modified release dosage form. As used herein, the term “modified release” refers to a dosage form in which the rate or place of release of the active ingredient(s) is different from that of an immediate dosage form when administered by the same route. Modified release dosage forms include delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof The release rate of the active ingredient(s) can also be modified by varying the particle sizes and polymorphorism of the active ingredient(s).

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; and 6,699,500, the contents of which are hereby incorporated by reference in their entirety.

5.3.1. Matrix Controlled Release Devices

The pharmaceutical compositions provided herein in a modified release dosage form can be fabricated using a matrix controlled release device known to those skilled in the art (see, Takada et al. in “Encyclopedia of Controlled Drug Delivery,” Vol. 2, Mathiowitz Ed., Wiley, 1999).

In one embodiment, the pharmaceutical compositions provided herein in a modified release dosage form is formulated using an erodible matrix device, which is water-swellable, erodible, or soluble polymers, including synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; and cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethylhydroxy ethylcellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

In further embodiments, the pharmaceutical compositions are formulated with a non-erodible matrix device. The active ingredient(s) is dissolved or dispersed in an inert matrix and is released primarily by diffusion through the inert matrix once administered. Materials suitable for use as a non-erodible matrix device included, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinyl acetate copolymers, ethylene/propylene copolymers, ethylene/ethyl acrylate copolymers, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, polyvinyl chloride, plasticized nylon, plasticized polyethylene terephthalate, natural rubber, silicone rubbers, polydimethylsiloxanes, silicone carbonate copolymers, and; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix controlled release system, the desired release kinetics can be controlled, for example, via the polymer type employed; the polymer viscosity; the particle sizes of the polymer and/or the active ingredient(s); the ratio of the active ingredient(s) versus the polymer, and other excipients or carriers in the compositions.

The pharmaceutical compositions provided herein in a modified release dosage form can be prepared by methods known to those skilled in the art, including direct compression, dry or wet granulation followed by compression, melt-granulation followed by compression.

5.3.2. Osmotic Controlled Release Devices

The pharmaceutical compositions provided herein in a modified release dosage form can be fabricated using an osmotic controlled release device, including one-chamber system, two-chamber system, asymmetric membrane technology (AMT), and extruding core system (ECS). In general, such devices have at least two components: (a) the core which contains the active ingredient(s); and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents water-swellable hydrophilic polymers, which are also referred to as “osmopolymers” and “hydrogels,” including, but not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents is osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol, organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof

Osmotic agents of different dissolution rates can be employed to influence how rapidly the active ingredient(s) is initially delivered from the dosage form. For example, amorphous sugars, such as MANNOGEM™ EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core can also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane can also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane can be formed post-coating by mechanical or laser drilling. Delivery port(s) can also be formed in situ by erosion of a plug of water-soluble material or by rupture of a thinner portion of the membrane over an indentation in the core. In addition, delivery ports can be formed during coating process, as in the case of asymmetric membrane coatings of the type disclosed in U.S. Pat. Nos. 5,612,059 and 5,698,220, the contents of which are hereby incorporated by reference in their entirety.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form can further comprise additional conventional excipients or carriers as described herein to promote performance or processing of the formulation.

The osmotic controlled-release dosage forms can be prepared according to conventional methods and techniques known to those skilled in the art. See, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27, the contents of which are hereby incorporated by reference in their entirety.

In certain embodiments, the pharmaceutical compositions provided herein are formulated as AMT controlled-release dosage form, which comprises an asymmetric osmotic membrane that coats a core comprising the active ingredient(s) and other pharmaceutically acceptable excipients or carriers. See, U.S. Pat. No. 5,612,059 and WO 2002/17918, the contents of which are hereby incorporated by reference in their entirety. The AMT controlled-release dosage forms can be prepared according to conventional methods and techniques known to those skilled in the art, including direct compression, dry granulation, wet granulation, and a dip-coating method.

In certain embodiments, the pharmaceutical compositions provided herein are formulated as ESC controlled-release dosage form, which comprises an osmotic membrane that coats a core comprising the active ingredient(s), a hydroxylethyl cellulose, and other pharmaceutically acceptable excipients or carriers.

5.3.3. Multiparticulate Controlled Release Devices

The pharmaceutical compositions provided herein in a modified release dosage form can be fabricated as a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates can be made by the processes known to those skilled in the art, including wet- and dry-granulation, extrusion/spheronization, roller-compaction, melt-congealing, and by spray-coating seed cores. See, for example, Multiparticulate Oral Drug Delivery; Marcel Dekker: 1994; and Pharmaceutical Pelletization Technology; Marcel Dekker: 1989.

Other excipients or carriers as described herein can be blended with the pharmaceutical compositions to aid in processing and forming the multiparticulates. The resulting particles can themselves constitute the multiparticulate device or can be coated by various film-forming materials, such as enteric polymers, water-swellable, and water-soluble polymers. The multiparticulates can be further processed as a capsule or a tablet.

### 5.4. Dosage

The pharmaceutical compositions that are provided can be administered for prophylactic and/or therapeutic treatments. An “effective amount” refers generally to an amount that is a sufficient, but non-toxic, amount of the active ingredient (i.e., a selective NTS2 compound) to achieve the desired effect, which is a reduction or elimination in the severity and/or frequency of symptoms and/or improvement or remediation of damage. A “therapeutically effective amount” refers to an amount that is sufficient to remedy a disease state or symptoms, or otherwise prevent, hinder, retard or reverse the progression of a disease or any other undesirable symptom. A “prophylactically effective amount” refers to an amount that is effective to prevent, hinder or retard the onset of a disease state or symptom.

In general, toxicity and therapeutic efficacy of the <NAME> can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compositions that exhibit large therapeutic indices are preferred.

The data obtained from cell culture and/or animal studies can be used in formulating a range of dosages for humans. The dosage of the active ingredient typically lines within a range of circulating concentrations that include the ED50 with little or no toxicity. The dosage can vary within this range depending upon the dosage form employed and the route of administration utilized.

The effective amount of a pharmaceutical composition comprising a selective NTS2 compound to be employed therapeutically or prophylactically will depend, for example, upon the therapeutic context and objectives. One skilled in the art will appreciate that the appropriate dosage levels for treatment, according to certain embodiments, will thus vary depending, in part, upon the molecule delivered, the indication for which the <NAME> is being used, the route of administration, and the size (body weight, body surface or organ size) and/or condition (the age and general health) of the patient. A clinician may titer the dosage and modify the route of administration to obtain the optimal therapeutic effect. Typical dosages range from about 0.1 μg/kg to up to about 100 mg/kg or more, depending on the factors mentioned above. In certain embodiments, the dosage may range from 0.1 μg/kg up to about 150 mg/kg; or 1 μg/kg up to about 100 mg/kg; or 5 μg/kg up to about 50 mg/kg.

The dosing frequency will depend upon the pharmacokinetic parameters of the <NAME> in the formulation. For example, a clinician will administer the composition until a dosage is reached that achieves the desired effect. The composition may therefore be administered as a single dose or as two or more doses (which may or may not contain the same amount of the desired molecule) over time, or as a continuous infusion via an implantation device or catheter. Treatment may be continuous over time or intermittent. Further refinement of the appropriate dosage is routinely made by those of ordinary skill in the art and is within the ambit of tasks routinely performed by them. Appropriate dosages may be ascertained through use of appropriate dose-response data.

Unless defined otherwise, all technical and scientific terms used herein have the same meaning as commonly understood by one of ordinary skill in the art to which this invention belongs. The article “a” and “an” are used herein to refer to one or more than one (i.e., to at least one) of the grammatical object(s) of the article. By way of example, “an element” means one or more elements.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The following Examples further illustrate the invention and are not intended to limit the scope of the invention. In particular, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

### 6. EXAMPLES

Several important milestone compounds from NT receptor research are depicted in Chart 1. The NT(8-13) fragment of NT, compound 1, is as potent as the full length peptide³⁰ and several hexapeptide variants of 1 have been reported over the years that either favor NTS2 over NTS1 or are selective for NTS2 versus NTS1. The first of these is JMV-431 (2) that was produced via reduction of the tyrosine 11 amide bond and shows a clear preference for NTS2^{31, 32}. While it is not bioavailable, it is active in several models of chronic pain when dosed intrathecally^{27, 28}. The peptide NT79 (3), reported more recently by Boules et al.³³, is selective for NTS2 (>100-fold) and provided a wealth of information regarding the role of NTS2 in animal models of pain, anti-psychotic activity, thermoregulation and regulation of blood pressure³³. Like peptide 2, this compound attained NTS2 selectivity via modification of the tyrosine 11 residue. The most recent additions to the rolls of NTS2 selective compounds are the potent peptide-peptoid hybrids 4a and 4b^{34, 35}. These compounds are ultra-selective for NTS2 with selectivity ratios reaching 12,000 and 22,000-fold respectively and with 4b also demonstrating excellent plasma stability.

Though few in number, there are three prominent non-peptide compounds that interact with NTS2 that have been used extensively in the characterization of the NT receptors. These include the two pyrazole-based compounds SR48692 (5a) and SR142948 (5b) and the histamine blocker levocabastine (6)³⁶⁻³⁸. Pyrazole compound 5a prefers NTS1 to NTS2 while 5b is non-selective but they both antagonize the activity of NT at NTS1. Compound 6 is selective for NTS2 versus NTS1 but it is also a potent antagonist at histamine receptor 1 (H1). These compounds (5a, 5b and 6) highlight the fact that while NTS2 selective peptides exist, selective non-peptides compounds have yet to be identified.

The paucity of selective non-peptide compounds suggested to us that screening efforts at the NTS2 receptor had either not been attempted or had never been reported despite that fact that the receptor had been reportedly expressed in numerous cell lines. We imagined that this could have arisen from the literature reports of NTS2 experiments that yielded seemingly contradictory data from the heterologously expressed NTS2 receptor. Indeed, NT has been reported to be an agonist, an inverse agonist and a neutral antagonist depending upon the expression system^{39-42.} Similar findings were reported for compounds 5a, 5b and 6 as well when tested alongside NT in these systems. Specifically, the potent NTS1 antagonists 5a and 5b were found to be agonists at NTS2 as they mobilized calcium release at NTS2 while levocabastine (6) was found to be a weak partial inverse agonist^{39, 41, 42}.

At the front end of our effort to find novel NTS2 active compounds, the actual biological disposition of NT was of less than concern than identifying a means of screening compound libraries. From this perspective, we viewed the reports above as an opportunity to accomplish this task. Working on the hypothesis that the calcium release described in these reports was NTS2 mediated, we studied the calcium release produced by the non-peptide compounds 5a and 5b in a CHO cell line expressing rNTS2. Using a FLIPR® tetra system, we examined analogs of 5a measuring their ability to either stimulate release of calcium or to block the calcium release stimulated by 5b and found that it responded to changes in structure. In this manner, we were able to identify, both directly and indirectly, those compounds that interact with NTS2 as well as determine activities relative to 5b.

We also compared the data collected from the FLIPR assay to the binding affinity data in competition with ¹²⁵I-NT for a set of analogs of 5a. This study revealed a positive correlation between binding affinity and calcium modulation for the NTS2 receptor; compounds that modulated calcium release also competed with NT for binding at NTS2. Using this method we have screened a variety of compound libraries and have identified a number of non-peptide NTS2 selective compounds. In this report we provide the details of our effort that led to the identification of a novel NTS2 selective potent partial agonist 1-({[1-(4-fluorophenyl)-5-(2-methoxyphenyl)-1H-pyrazol-3-yl]carbonyl}amino)cyclohexane carboxylic acid (NTRC-739, 7b) starting from a nonselective full NTS2 agonist compound 5a (SR48692).

Chemistry Section

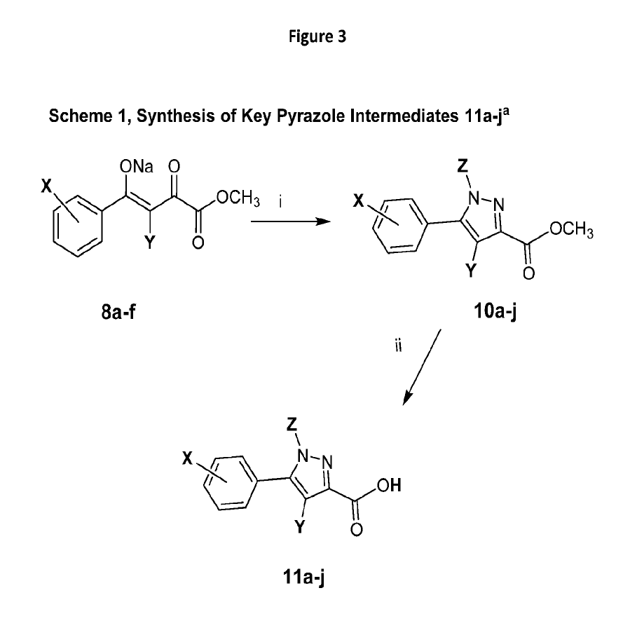

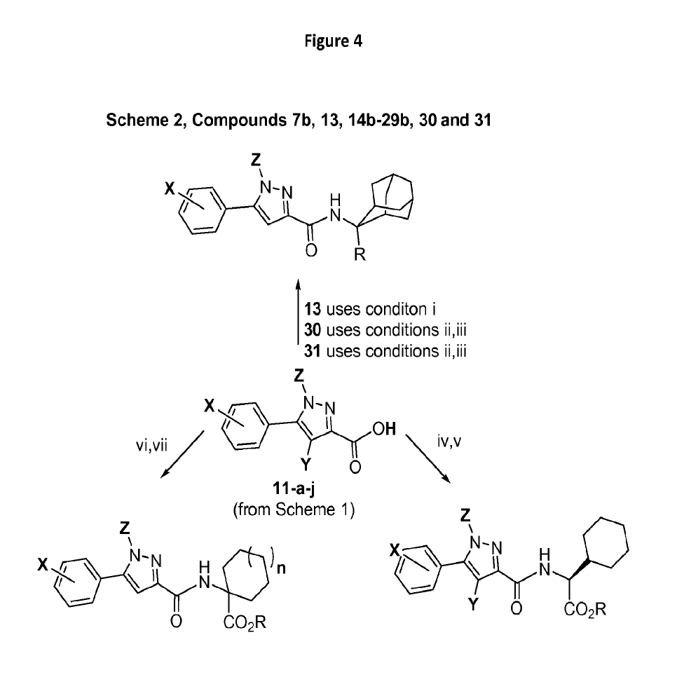

The target compounds, depicted in Tables 2 and 3, were synthesized as described in Schemes 1-2 (FIG. 3 and FIG. 4). Elemental analysis for key compounds appears in Table 5. Commercially available materials were used where possible. Those not commercially available were prepared according to literature precedent. Scheme 1 illustrates the synthesis of the key intermediate pyrazole carboxylic acids (11a-j). These were prepared as described by Labeeuw⁴³, though improved methods were recently described by Jiang et al.⁴⁴ and Baxendale et al.⁴⁵ This employed a Knorr [3+2]-cyclization reaction between 4-aryl-2,4-diketoesters (8a-f) and arylhydrazines 9a-c in acetic acid at reflux. The resulting esters 10a-j were hydrolyzed using LiOH and dioxane to give 11a-j. The 4-aryl-2,4-diketoesters were commercially available with the exception of 8b. The 2,6-dimethoxy-butyrophenone used to synthesize compound 8b was prepared exactly as described by Lindh⁴⁶.

In Scheme 2 the synthetic methods used to produce target compounds 7b, 13, 14b-29b, 30 and 31 are described. Thus, a given pyrazole carboxylic acid (11a-j) and amino acid ester (12a-d) or amine (12e) from Chart 2 were coupled using O-benzotriazol-1-yl-N,N,N′,N′-tetramethyluronium hexafluorophosphate (HBTU) and triethylamine to give ester intermediates 7a,14a-29a and the descarboxy target compound 13. The ester intermediates 7a, 14a-29a were hydrolyzed to give final products using either basic (LiOH and dioxane) or acidic (trifluoroacetic acid (TFA) in CH₂Cl₂) conditions as shown. We used the tert-butyl rather than the methyl esters of L-cyclohexylglycine (12d) to avoid racemization of the chiral amino acid. We used the basic conditions for hydrolysis of achiral amino acid esters. The synthesis of target compounds 30 and 31 was accomplished according to the method of Quéré⁴⁷ wherein pyrazole acids 11i and 11j were converted to the acid chloride using SOCl₂ in toluene, followed by coupling of the acid chloride intermediate with the adamantyl amino acid 12f under Schotten-Bauman conditions. The adamantyl amino acid 12f was prepared exactly as described by Nagasawa⁴⁸.

Biological Results

The binding affinities of the test compounds for the rNTS1 and rNTS2 receptors listed in Tables 1 through 4 were determined using previously reported competitive binding assays⁴⁹. The NTS1 and 2 receptors were labeled using ¹²⁵I-NT. The cells used in the binding assay were CHO-kl cells (American Type Culture Collection) engineered to over-express either the rNTS1 or rNTS2 receptor. Measures of functional agonism and antagonism were obtained by measuring changes in intensity of a calcium-sensitive fluorescent dye as an indirect measure of changes in internal calcium concentrations. These measurements were performed using a FLIPR Tetra (NTS2) or a FlexStation II plate reader for NTS1 (Molecular Devices) and were analyzed using GraphPad Prism software. Antagonism was measured as inhibition of NT-induced calcium release (NTS1) or inhibition of SR142948 (5b)-induced calcium release (NTS2).

Results and Discussion

The screening of compound libraries to identify starting points for new drug discovery is a common practice. The radioligand binding method of screening compound libraries presents a significant challenge as the size of a library is increased, as this requires significant amounts of an expensive radioligand. In addition, radioligand binding studies yield no information regarding the functional characteristics (agonist, antagonist etc.) of the hits. The FLIPR tetra system, on the other hand, is a functional assay that can be used to screen libraries quickly and cheaply using calcium release as an end point. The calcium release reported for compounds 5a and 5b in NTS2 cell lines presented an opportunity to harness this alternative technology to speed along discovery efforts for NTS2 selective non-peptide compounds. To determine the usefulness of such an approach, we analyzed the compounds commonly used in NT receptor research. In Table 1, we have summarized the calcium release and binding data obtained for NT, NT(8-13) (1), as well as the NTS1 antagonist pyrazole-based compounds 5a and 5b, and the NTS2 selective compound levocabastine (6) in both our NTS1 and NTS2 cell lines.

In our CHO-kl-NTS2 cell line neither NT nor the NT(8-13) fragment (1) showed any calcium release in our FLIPR assay. The NTS1 antagonists 5a and 5b, on the other hand, stimulated calcium release with EC₅₀ values of 120 and 20 nM, respectively. Compound 5b was more potent than compound 5a in both the calcium release and binding assays (described below) and was thereby designated as our NTS2 full agonist standard (the use of the term agonist here refers to a compound that stimulates calcium release in this assay). Levocabastine (6), a compound used for years to distinguish NTS1 from NTS2, was also tested and found to be a potent partial agonist with an EC₅₀ of 28 nM and an Eₘₐₓ of 16% relative to 5b.

While NT and NT(8-13) (1) did not stimulate calcium release, they both blocked the calcium release stimulated by compound 5b in an insurmountable manner as they showed a rightward shift in the dose response curve with an accompanying dose-dependent drop in the Eₘₐₓ value of 5b^{50, 51}. We thus determined and report the IC₅₀ for both NT and 1 in competition with 5b and found these to be 18.9 and 5.4 nM, respectively.

We also examined these compounds in a radioligand binding assay in the NTS2 cell line using ¹²⁵I-NT. We found that compound 5a gave a Kᵢ of 62 nM while 5b showed a ten-fold more potent Kᵢ of 6 nM. As in the FLIPR assay, compound 5b is more potent than 5a. NT gave a Kᵢ of 18.5 nM, similar to its Kᵢ for blocking the calcium release stimulated by compound 5b. NT(8-13) showed similar affinity to NT while levocabastine (6) gave a Kᵢ of 33 nM. The binding affinities found for NT, 1 and levocabastine were slightly higher than found in other laboratories while those for 5a and b are similar. We attribute this to our use of an in-plate whole cell binding method⁴⁹ as opposed to the use of membrane preparations.

In the NTS1 FLIPR assay, the activity of each of the reference compounds was found to be within expectation. Thus, NT and 1 showed potent stimulation of calcium release at NTS1 with EC₅₀ values of 0.04 and 0.01 nM, respectively while compounds 5a and b blocked NT mediated calcium release with Kₑ values of 4.7 and 1.5 nM, respectively. The NTS2 selective compound levocabastine (6) showed neither agonist nor antagonist activity toward NT-mediated calcium release, as expected.

The data obtained from the testing of these reference compounds suggested that this calcium release assay would be useful for detecting NTS2 mediated activity in novel compounds. First, each compound that either stimulated or blocked calcium release in this NTS2 cell line also possessed the ability to compete with NT for binding. This implies that the calcium release observed in this assay is the result of interaction between the test compound and the NTS2 receptor. This assertion was strengthened by the fact that these same compounds produce no effects in the parent CHO-kl cells not transfected with the NTS2 receptor (data not shown). Third, the assay proved capable of identifying a range of activities from potent agonist (5a and b) to partial agonist (6) to antagonist (NT and 1) implying that the structure of the compound made a direct impact on the resulting mobilization of calcium.

In a separate experiment, we compared the activity of compound 5a to its descarboxy derivative 13 (Scheme 1) as the carboxyl group is known to be a primary attachment point for ligands of the neurotensin receptors⁵²⁻⁵⁴ We found that compound 13 was inactive in both NTS1 and NTS2 FLIPR assays (Table 1) and gave a Kᵢ>11 uM in the radioligand binding assay. Thus, the behavior of 13 stands in stark contrast to the activity found for 5a and provided additional evidence that the calcium release observed for 5a (and 5b) results from these ligands interacting with NTS2.

As described earlier, NT possesses neuroleptic activity and mediates non-opioid analgesia while both NT and levocabastine (6) are active in models of chronic pain^{28, 33}. The pyrazole compounds 5a and 5b, on the other hand, are not analgesics in vivo but instead block the analgesic activity of NT in animal models of pain^{27, 55, 56}. This is also observed in animal models based on runaway mesolimbic dopamine where the neuroleptic action of NT is blocked by compounds 5a and 5b^{20, 37, 57, 58}. The data (Table 1) showed that in our CHO-NTS2 expression system the proven analgesic and anti-psychotic compounds appear as either antagonists of calcium release (NT and peptide 1) or potent partial agonists of calcium release (levocabastine, 6). The compounds that possess neither analgesic nor anti-psychotic activity in vivo, compounds 5a and 5b, appeared as potent agonists of calcium release. This point of reference provided a means of parsing active compounds into two categories, those that might possess desirable behaviors in animal models and those that might not. Based on these two types of in vitro behaviors our search for novel compounds followed two pathways, one for NTS2 selective antagonists and one for NTS2 selective potent partial agonists.

In this document we provide a summary of the key SAR elements discovered while testing this calcium release assay that led to the identification of the potent partial agonist NTRC-739 (7b). Each of the appended molecular regions of 5a are discussed herein and include: the dimethoxyphenyl ring, the amino acid side chain, the 7-chloroquinoline ring, and the 4-position of the pyrazole ring. The data obtained from this representative set of compounds are provided in Tables 2 and 3 and include the NTS2 EC₅₀ as well the NTS1 Kₑ and the NTS2 binding affinity (Kᵢ). Also, in the preparation of compound libraries, we used the L-cyclohexyl glycine side chain seen in 14b (Table 2) as a surrogate for the adamantyl group in compound 5a as we found it easier to produce and isolate en masse. Compound 14b, like 5a, was reported to be a potent NTS1 antagonist by Quéré⁵⁹ and thus was expected to be an agonist in the NTS2 assay given the results obtained for 5a and 5b. The data from 14b indicated that it would be a suitable stand in for 5a as it provided comparable agonist potency and efficacy for NTS2 (EC₅₀ of 217 and Eₘₐₓ 86% of 5b) and NTS1 antagonist activity (Kₑ of 23 nM). The 10-fold difference in its relative binding affinity at NTS2 demonstrated that the structure of the amino acid side chain significantly impacts binding affinity.

A comparison of the data obtained for compounds 14b-18b (Table 2) illustrates the SAR realized from changing the position of the 6-methoxy group (14b, 15b, 16b) or from the elimination of the 6-methoxy group (17b) and from replacing the 2,6-methoxy groups with fluorine atoms (18b). The NTS1 antagonist activity fell off significantly across the series of positional isomers 14b-16b with Kₑ values of 23 nM, 1275 nM and >10 μM respectively. The same was seen for 17b and the di-fluoro derivative (18b) with Kₑ values of 1682 nM and >10 μM. The NTS2 efficacy of calcium release also fell across this series of compounds while the potency was little varied. In line with the latter, the binding affinities across this same series of compounds showed surprisingly little variation compared with the change seen going between 5a and 14b. As the amino acid side chain wasn't varied in 14b-18b, this information reinforced the notion that the structure of the amino acid side chain has a strong impact on binding affinity and appeared to control the range of activity. Overall, we found that the potency of antagonist activity for 14b at the NTS1 receptor relied heavily upon the 2,6-dimethoxyphenyl ring for its activity. At NTS2, it was the efficacy of calcium release that was most significantly affected by alteration of the 2,6-dimethoxyphenyl ring. The binding affinity data and NTS2 potency, on the other hand, appeared to move in line with one another and were much less affected by changes to this molecular region.

On the whole, compounds 14b-18b showed a shift towards behavior mimicking the NTS2 selective partial agonist levocabastine. This included significantly weakened antagonist activity at NTS1 (higher Kₑ) and lowered efficacy at NTS2 (Eₘₐₓ) with little impact on NTS2 potency (EC₅₀) leaning toward improved NTS2 selectivity. However, the low NTS2 affinity and potency of these compounds suggested that alternative structural changes would be required to more closely align with levocabastine.

The data from compound 19b illustrated the effect on in vitro activity produced by alkylation of the 4-position of the pyrazole ring. The changes seen here resembled the previous set of compounds as the NTS1 antagonist activity fell off significantly relative to 14b with a Kₑ shift from 23 nM to >10 μM and the NTS2 efficacy was also decreased with Eₘₐₓ values of 86 for 14b to 15% of 5b for 19b. The NTS2 potency was also decreased compared with 14b (EC₅₀ values of 120 and 94 nM for 14b and 19b respectively) and the binding affinity of 19b also fell in the range of the previous set of compounds presumably because it bore the L-cyclohexylglycine side chain. The fact that the structural change in 19b provided data that appeared to be an extension of the SAR seen in the changes to the dimethoxyphenyl ring is likely related to the close proximity of these two structural features within the molecule. This, in turn, suggests that the ethyl group could be preventing rotation of the phenyl ring or that it could be interfering with a cation-n bond. Irrespective of mechanism, this example illustrates that alkylation of this position provided compounds that favored NTS2 activity over NTS1 but with unacceptably low levels of affinity at NTS2.

As seen above, the amino acid side chain in compounds like 5a is known to have a strong influence on ligand behavior presumably due to its proximity to the carboxyl group, the primary anchoring point of the ligand to the receptor. We observed that compounds with large achiral alicyclic side chains generally had good potency at NTS2 while linear or branched aliphatic chains were much weaker at NTS2 and showed partial agonist activity at NTS1⁶⁰. Unfortunately, compounds with large achiral alicyclic side chains were not selective for NTS2 as they also possess good NTS1 antagonist activity. The data obtained for compounds 5a, 14b and 20b-23b in Table 2 illustrate the SAR typical of the large cyclic amino acid side chains. This group of compounds, each possessing the 2,6-dimethoxyphenyl ring and the 7-chloroquinoline group, demonstrated that large cyclic (cycloheptyl, 20b), large bicyclic (adamantyl, 5a), and large pendant alicyclic (14b) structures are potent NTS1 antagonists with Kₑ values of 42, 4.7 and 23 nM respectively. Compounds with smaller cyclic rings were somewhat less potent at NTS1, Kₑ=222 nM for the five-membered cyclic ring (22b) and 157 nM for the six-membered cyclic ring (21b). According to these data then, the compounds with smaller cyclic rings (21b, 22b) trended toward NTS2 selectivity. Compounds 20b-22b also showed increased NTS2 agonist potency (lower EC₅₀) but this was accompanied by full efficacy (high Eₘₐₓ relative to 5b) at NTS2, exactly opposite of that found in levocabastine (6).

In their favor, however, these same compounds (20b-22b) showed improved binding affinity at NTS2 versus 14b (Kᵢ=170, 151 and 102 nM versus 644 nM for 14b), more in line with 5a (Kᵢ=62 nM). We found this binding affinity and potency improvement worked in concert with the lowered NTS1 activity observed for the compounds above that lacked the 2,6-dimethoxy substitution (15b-18b). This provided enhanced NTS2 affinity and potency while maintaining low efficacy and lowered NTS1 activity. A comparison of the data from the compound couple, 21b and 23b, to the data from the compound couple 14b and 17b illustrates this point. The 2-methoxyphenyl substituted compounds, 23b and 17b, showed NTS1 antagonist activity that was considerably lower than that found for the 2,6-dimethoxyphenyl substituted analogs 21b and 14b. The NTS2 potency (EC₅₀) was either unchanged or improved and both sets showed an accompanying decrease in NTS2 efficacy (Eₘₐₓ). The observed improvement in NTS2 binding affinity for compound 23b versus 17b thus demonstrates the contribution of the amino acid side chain.

This comparison of NTS2 agonist potency to the NTS1 antagonist potency for compounds 21b and 23b was an important clue to achieving selectivity in this series of compounds as this data reinforced the data from the previous series which showed that the NTS1 receptor relied much more heavily upon the 2,6-dimethoxyphenyl ring for its activity compared with NTS2 and that the amino acid side chain could work in concert with the methoxyphenyl ring to retain these desired properties. This trend was further advanced in compounds that did not possess the chloroquinoline group. In Table 3, two alternate substitutions for the chloroquinoline group, the napthyl (A) and 4-fluorophenyl (B) are depicted along with their associated data. These substitutions for the 7-chloroquinolyl group were included in our compound libraries during their synthesis because the napthyl compounds were known to yield NTS1 antagonists⁴⁷ and the 4-fluorophenyl group was found in levocabastine (6).

Like their 7-chloroquinolyl counterparts, the napthyl-substituted 2,6-dimethoxyphenyl derivatives 24b and 26b possess potent NTS1 antagonist activity and also NTS2 agonist activity, however, distinct differences were found as well. Most notably, the Kₑ values for NTS1 antagonist activity and the NTS2 Eₘₐₓ values were roughly half of that found for similarly substituted chloroquinoline-based compounds. These compounds were inherently less potent at NTS1 and less efficacious at NTS2. This was observed for both the L-cyclohexyl glycine (24b) and the 1-aminocyclohexancarboxylic acid side chains (26b). Comparing the napthyl (24b) and the 7-chloroquinolyl (14b) compounds (Tables 2 and 3), we found Kₑ values of 58 versus 23 nM and NTS2 Eₘₐₓ values of 45 and 86% of 5b respectively. Comparing compounds 26b and 21b, we found Kₑ values of 230 versus 157 nM and NTS2 Eₘₐₓ values of 35 and 78% of 5b respectively.

As expected from the 7-chloroquinolyl series, the napthyl-substituted 2-methoxyphenyl derivatives 25b and 27b were less potent NTS1 antagonists than their 2,6-dimethoxyphenyl substituted counterparts. More surprising was the observation that the NTS2 efficacies for these compounds were nearly half again as low as that found in similarly substituted 7-chloroquinolyl compounds 17b and 23b, a second example of cooperative behavior or SAR working in concert to provide additive results. This is readily revealed in a comparison of the NTS2 efficacy data obtained for the 2,6-dimethoxy, 7-chloroquinolyl compound 21b, the 2-methoxy 7-chloroquinolyl 23b and the 2-methoxy napthyl substituted compound 27b with NTS2 Eₘₐₓ values of 78, 35, and 18% of 5b respectively.

There were also changes observed in the NTS2 EC₅₀ and binding affinity (Kᵢ) data for the napthyl-substituted versus the 7-chloroquinolyl-substituted pyrazole compounds. Napthyl-substituted compounds, bearing the 2,6-dimethoxyphenyl ring were more potent than their chloroquinoline-based counterparts (14b, 17b, 21b and 23b) but, napthyl compounds bearing the 2-methoxyphenyl ring were equally potent. This was observed for compounds with the L-cyclohexyl glycine as well as those with 1-aminocyclohexancarboxylic acid as in 26b and 27b. The NTS2 binding data observed for the napthyl-substituted compounds 24b-27b was found to be in line with that seen for the similarly substituted 7-chloroquinolyl-substituted compounds with the 2-methoxy derivatives showing lower affinity (higher Kᵢ values) than the 2,6-dimethoxy counterparts. As before though, the compounds bearing the L-cyclohexyl glycine side chain (24b,25b) were less potent than those with the 1-aminocyclohexancarboxylic side chain (26b,27b).

At the NTS2 receptor, the calcium data profile for compound 26b looked promising as it was found to be moving closer to levocabastine (6). Compound 26b showed two-fold greater NTS2 potency and comparable efficacy. The binding data was also similar but in this case, compound 6 was more potent showing a Kᵢ 3.5-fold greater than that for 26b. The similarity between these two compounds ended however when comparing receptor selectivity as compound 26b showed substantial NTS1 antagonist activity while levocabastine (6) displayed no activity at NTS1. The 2-methoxy napthyl-substituted analog 27b was not able to compensate for this deficit for while its NTS1 antagonist activity was lowered by twenty-fold, the NTS2 potency and binding affinity also suffered substantial losses.

Compounds bearing a 4-fluorophenyl-substituent (30, 31, 28b, 29b, 7b) are shown in Table 3. Comparison of the adamantyl-substituted compounds 5a and 30 and 31 highlights the contribution of the 4-fluorophenyl ring. These data show that the NTS1 antagonist activity was diminished by 40-fold for 30 versus 5a with Kₑ values of 191 and 4.7 nM respectively. The NTS2 potency, on the other hand, was doubled (EC₅₀ values of 120 versus 68 nM) while the efficacy was less than half relative to 5a (Eₘₐₓ values of 100 versus 34% of 5b). This phenomenon was also observed in the data for compounds 28b, 29b and 7b. These compounds are all far less active at NTS1 compared with their corresponding 7-chloroquinolyl (14b, 21b and 23b) or napthyl (24b, 26b and 27b) analogs and thereby attain enhanced NTS2 selectivity with respect to calcium release. The 4-fluorophenyl-substituted compounds (29b and 7b), bearing the 1-aminocyclohexancarboxylic acid substituent, showed the most levocabastine-like profiles whether they had a 2,6-dimethoxyphenyl ring (29b) or a 2-methoxyphenyl ring (7b). In keeping with previous observations, the transition to the 2-methoxyphenyl ring (7b) from the 2,6-dimethoxyphenyl ring (29b) led to a nearly 50% reduction in NTS2 efficacy with NTS2 Eₘₐₓ values of 15 and 8% of 5b respectively.

Their NTS2 binding affinity was also consistent with similarly substituted 7-chloroquinolyl (21b, 23b) or napthyl-substituted (26b, 27b) analogs bearing the 1-aminocyclohexanecarboxylic acid substitution with one important exception, the binding affinity did not decrease in going from the 2,6-dimethoxyphenyl (29b) to the 2-methoxyphenyl ring substitution (7b) with Kᵢ values of 140 and 153 nM respectively. We imagine that this change in SAR for 29b and 7b could result from a change in binding mode at NTS2 that is permitted for 4-fluorophenyl but not 7-chloroquinolyl or napthyl-substituted analogs. But whatever the reason, the 4-fluorophenyl-substitution was able to overcome the deficits found in both the 7-chloroquinolyl and napthyl-substituted analogs to provide pyrazole-based compounds (29b and 7b) with enhanced NTS2 potency and binding affinity with significantly lower efficacy compared to 5a at the NTS2 receptor.

From the standpoint of calcium mobilization at NTS1 and NTS2, compounds (29b and 7b) appear to be selective for the NTS2 receptor. However, we were not certain that this was a fair comparison as one assay measures calcium release (NTS2) and the other blockade of calcium release (NTS1). We therefore acquired the radioligand binding data for both 29b and 7b at NTS1 in order to compare like measurements. As seen in Table 4, comparison of the relative binding affinities at NTS1 and NTS2 showed that compound 7b is 161-fold selective for NTS2 versus NTS1 while the dimethoxy analog 29b shows a 23-fold preference for NTS2 over NTS1.

The identification of 7b and 31 as an NTS2 selective compounds demonstrated that this calcium assay was useful for driving SAR studies in the pyrazole carboxamide series of compounds. However the low efficacy of 7b, less than half of that found for levocabastine, prompted us to revisit mobilization of calcium release in the parent CHO cell line. We thus carried a final study and examined all of the compounds reported herein that showed NTS2 Eₘₐₓ values less than 15% of 5b. This was done collectively comparing all of the low efficacy compounds together in the same assay, under identical conditions, in our CHO-kl-NTS2 cells and simultaneously in the parent CHO line. The results of these experiments confirmed that the calcium mobilization observed was only found in the CHO cells stably expressing the NTS2 receptor.

In summary, we have tested numerous analogs of 5a in a calcium mobilization assay and in comparison with binding affinity data and have determined that it correctly identifies compounds that are active at NTS2. In SAR studies with 5a we identified patterns of behavior for each of the appended molecular regions including the: the dimethoxyphenyl ring, the amino acid side chain, the 7-chloroquinoline ring, and the 4-position of the pyrazole ring. We found that changes to the 2,6-dimethoxyaryl ring significantly decreased NTS1 activity and NTS2 efficacy of calcium release while leaving NTS2 potency and binding affinity little changed. Alkylation of the 4-position with an ethyl group provided data nearly indistinguishable from that obtained by changing substitution pattern of the 2,6-dimethoxyaryl ring. We found the amino acid side chain has a powerful influence on ligand affinity and potency at NTS2. Furthermore, the large cyclic or appended alicylic groups provided potent NTS1 compounds with full efficacy at NTS2 while smaller cyclic rings favored NTS2 over NTS1 in potency and binding. We also established that the potency and affinity enhancement discovered with the smaller cyclic rings was preserved with a change from a 7-chloroquinolyl to 4-fluorphenyl group. This provided compounds with both lower NTS2 efficacy and enhanced NTS2 selectivity and led us to the discovery of 7b and 31, as pyrazole-based compounds with properties similar to levocabastine in this assay.

### CONCLUSIONS

We have determined that the NTS2 mediated calcium release described by other researchers for SR48692 (5a) and SR142948 (5b) can be applied to the discovery of novel NTS2 active compounds. We have tested many of the compounds common to neurotensin research accordingly and have identified their associated calcium mobilization patterns in this assay. We found that compounds known to possess analgesic and anti-psychotic activity in vivo appear as antagonists (NT and NT(8-13)) or potent partial agonists (levocabastine). Using this NTS2 calcium mobilization assay as a guide to SAR, we have demonstrated that it is possible to lower the efficacy profile of 5a to produce compounds with in vitro profiles more closely aligned with levocabastine (6). Using this assay in combination with a FLIPR assay for NTS1, we were able to identify compounds 7b and 31 that are selective for NTS2 versus NTS1. Radioligand binding experiments carried out on the compounds described herein revealed a positive correlation between binding affinity at NTS2 versus ¹²⁵1-NT and NTS2 mediated calcium mobilization. Comparison of the Kᵢ data obtained for 7b and 31 from both the NTS2 and NTS1 binding assays provided additional confirmation of their selectivity.

We are currently working to correlate our in vitro results to specific actions in vivo using appropriate animal models. Additionally, compound 7b is being tested to determine if it is active as an H1 antagonist like levocabastine. We recognized at the outset that identification of a pyrazole-based levocabastine-like compound would provide an opportunity to achieve selectivity versus the H1 receptor since it lacks a basic amine, which is common to both the H1 histamine pharmacophore and levocabastine (6). Also see, Thomas et al., 2014 J. Med Chem 57 5318-5332, the contents of which are hereby incorporated by reference in its entirety.

Experimental Section

Reactions were conducted under a nitrogen atmosphere using oven-dried glassware as required. All solvents and chemicals used were reagent grade. Anhydrous tetrahydrofuran (THF), dichloromethane (DCM), and N,N-dimethylformamide (DMF) were purchased from VWR and used without further purification. Unless otherwise mentioned, all reagents and chemicals were purchased from commercial vendors and used as received. Flash column chromatography was carried out using a Teledyne ISCO Combiflash Rf system and Redisep Rf gold pre-packed HP silica columns. Purity and characterization of compounds were established by a combination of HPLC, TLC, mass spectrometry (MS), elemental analysis and NMR analytical techniques described below. ¹H and ¹³C NMR spectra were recorded on a Bruker Avance DPX-300 (300 MHz). Chemical shifts are reported in ppm relative to the tetramethylsilane, and coupling constant (J) values are reported in Hertz (Hz). Low-resolution mass spectra were obtained using a Waters Alliance HT/Micromass ZQ system (ESI). Thin layer chromatography (TLC) was performed on EMD precoated silica gel 60 F₂₅₄ plates, and spots were visualized with UV light and 12 or phosphomolybdic acid stain. CHO-kl cells were from the American Type Culture Collection, ¹²⁵I-neurotensin was from Perkin-Elmer, Calcium 5 Dye was from Molecular Devices, cell culture reagents were from Life Technologies.

### General Method for Preparation of Pyrazole Carboxylic Acid Esters (10a-j)

A magnetically stirred solution of a 4-aryl-2,4-diketoester sodium salt (8a-f, 5 mmol) and arylhydrazine hydrochloride (9a-c, 5 mmol) in glacial acetic acid (35 mL) and conc. HCl (1.5 mL) was heated under reflux for 5 hours and then cooled to rt. This was then poured into 300 mL of water and extracted five times with CH₂Cl₂. The extracts were washed carefully with sat'd NaHCO₃ and then water and brine then dried over Na₂SO₄ and concentrated to give a crude material. This material was purified using flash chromatography to give methyl esters 10a-j as foamy solids upon removal of solvent.

### General Method for Preparation of Pyrazole Carboxylic Acids (11a-j)

Pyrazole carboxylic acids 11a-j were prepared from the esters (10a-j) using the Methyl Ester Hydrolysis Method described below.

### General Amide Coupling

To a magnetically stirred suspension of the appropriate pyrazole carboxylic acid (11a-j, 0.122 mmol) in anhydrous CH₂Cl₂ (20 mL) were added successively triethylamine (0.366 mmol), HBTU (0.146 mmol), and the appropriate amino acid ester hydrochloride salt (12a-e, 0.122 mmol). After stirring for 16 h, the resulting mixture was concentrated and the residue purified by flash chromatography using a gradient of 0-100% EtOAc in hexanes to give an intermediate ester that was taken directly to the hydrolysis step.

### General Methyl Ester Hydrolysis

To a magnetically stirred solution of methyl ester (7a,20-23a,26a,27a,29a) from the coupling reaction (0.1 mmol) in 1,4-dioxane (5 mL) was added 1N LiOH (2 mL) followed by stiffing at room temperature overnight. The mixture was then concentrated and the residue was taken up in water (2 mL) and extracted with ethyl acetate (15 mL). After this, 2N HCl was added to the aqueous layer to precipitate the carboxylic acid final product. This was extracted twice with CH₂Cl₂ and the combined extracts were dried over Na₂SO₄, and concentrated to give solid final products.

### General tert-Butyl Ester Hydrolysis

To a magnetically stirred solution of the tert-butyl ester intermediate (14-19a,24a,25a,28a) obtained from the coupling reaction (0.08 mmol) in CH₂Cl₂ (10 mL) was added excess TFA (10 mL) at room temp. After stiffing for 16 h, the mixture was concentrated and then triturated with ethyl ether to give a solid product that was isolated by vacuum filtration, washed with ether and then dried under high vacuum overnight.

### Methyl 1-(7-chloroquinolin-4-yl)-5-(2,6-dimethoxyphenyl)-1H-pyrazole-3-carboxylate (10a)

Ester 10a was prepared via the general procedure starting from methyl 4-(2,6-dimethoxyphenyl)-2,4-dioxobutanoate sodium salt (8a) and 7-chloro-4-hydrazinylquinoline hydrochloride (9a) to give 10a (68%). ¹H NMR (CDCl₃) δ 8.76 (d, J=4.71 Hz, 1H), 8.10 (s, 1H), 7.89 (d, J=9.04 Hz, 1H), 7.48 (td, J=1.04, 9.04 Hz, 1H), 7.16-7.30 (m, 2H), 7.02-7.14 (m, 1H), 6.40 (d, J=8.48 Hz, 2H), 4.39-4.53 (m, 2H), 3.42 (s, 6H), 1.42 (dt, J=0.94, 7.16 Hz, 3H).

### Methyl 1-(7-chloroquinolin-4-yl)-5-(2,6-dimethoxyphenyl)-4-ethyl-1H-pyrazole-3-carboxylate (10b)

Ester 10b was prepared from methyl 3-[(2,6-dimethoxyphenyl)carbonyl]-2-oxopentanoate, sodium salt (8b) and 7-chloro-4-hydrazinylquinoline hydrochloride (9a) according to the general method (45%). ¹H NMR (CDCl₃) δ 7.69 (d, J=8.4 Hz, 2H), 7.11-7.00 (m, 3H), 6.93-6.79 (m, 4H), 6.68 (d, J=7.5 Hz, 1H), 6.64-6.56 (m, 2H), 6.36 (d, J=8.2 Hz, 1H), 5.59-4.69 (m, 3H), 4.19-4.00 (m, 1H), 3.72 (q, J=7.0 Hz, 1H), 3.48 (d, J=0.8 Hz, 1H), 2.95 (br. s., 1H), 2.91-2.79 (m, 3H), 2.76-2.65 (m, 1H), 2.62-2.49 (m, 1H), 2.08-1.88 (m, 1H), 1.67 (br. s., 2H), 1.29 (s, 3H), 0.95 (t, J=6.4 Hz, 6H).

### Methyl 1-(7-chloroquinolin-4-yl)-5-(2,5-dimethoxyphenyl)-1H-pyrazole-3-carboxylate (10c)

Ester 10c was prepared from methyl 4-(2,5-dimethoxyphenyl)-2,4-dioxobutanoate sodium salt (8c) and 7-chloro-4-hydrazinylquinoline hydrochloride (9a) according to the general method (57%). ¹H NMR (CDCl₃) δ 8.77 (d, J=4.52 Hz, 1H), 8.14 (d, J=2.07 Hz, 1H), 7.91 (d, J=9.04 Hz, 1H), 7.54 (dd, J=1.98, 8.95 Hz, 1H), 7.14 (s, 1H), 7.01 (d, J=4.52 Hz, 1H), 6.78-6.91 (m, 2H), 6.57 (d, J=8.85 Hz, 1H), 3.99 (s, 3H), 3.73 (s, 3H), 2.91 (s, 3H).

### Methyl 1-(7-chloroquinolin-4-yl)-5-(2,4-dimethoxyphenyl)-1H-pyrazole-3-carboxylate (10d)

Ester 10d was prepared from methyl 4-(2,4-dimethoxyphenyl)-2,4-dioxobutanoate sodium salt (8d) and 7-chloro-4-hydrazinylquinoline hydrochloride (9a) according to the general method (45%). ¹H NMR (CDCl₃) δ 8.78 (d, J=4.62 Hz, 1H), 8.14 (d, J=1.79 Hz, 1H), 7.87 (d, J=8.95 Hz, 1H), 7.52 (dd, J=1.98, 9.04 Hz, 1H), 7.20 (d, J=8.38 Hz, 1H), 7.09 (s, 1H), 7.02 (d, J=4.62 Hz, 1H), 6.47 (dd, J=2.21, 8.43 Hz, 1H), 6.19 (d, J=2.07 Hz, 1H), 3.98 (s, 3H), 3.77 (s, 3H), 2.99 (s, 3H).

### Methyl 1-(7-chloroquinolin-4-yl)-5-(2,6-difluorophenyl)-1H-pyrazole-3-carboxylate (10e)

Ester 10e was prepared from methyl 4-(2,6-difluorophenyl)-2,4-dioxobutanoate sodium salt (8e) and 7-chloro-4-hydrazinyl-quinoline hydrochloride (9a) according to the general method (52%). ¹H NMR (CDCl₃) δ 8.87 (d, J=4.52 Hz, 1H), 8.14 (d, J=1.88 Hz, 1H), 7.61-7.75 (m, 1H), 7.51 (dd, J=1.88, 9.04 Hz, 1H), 7.41 (dd, J=0.94, 8.67 Hz, 1H), 7.25-7.35 (m, 1H), 7.22 (d, J=4.52 Hz, 1H), 7.06-7.15 (m, 1H), 6.77-6.89 (m, 2H), 4.0 (s, 3H). ***MS? Anal.?

### Methyl 1-(7-chloroquinolin-4-yl)-5-(2-methoxyphenyl)-1H-pyrazole-3-carboxylate (10g)

Ester 10f was prepared from methyl 4-(2-methoxyphenyl)-2,4-dioxobutanoate sodium salt (80 and 7-chloro-4-hydrazinylquinoline hydrochloride (9a) according to the general method (43%). ¹H NMR (CDCl₃) δ 8.78 (d, J=4.71 Hz, 1H), 8.15 (s, 1H), 7.91 (d, J=9.04 Hz, 1H), 7.5 (td, J=1.04, 9.04 Hz, 1H), 7.27-7.32 (m, 3H), 6.96-7.01 (m, 2H), 6.65 (d, J=8.48 Hz, 2H), 4.0 (s, 3H), 4.0 (s, 3H).

### Methyl 5-(2,6-dimethoxyphenyl)-1-naphthalen-1-yl-1H-pyrazole-3-carboxylate (10g)

Ester 10g was prepared from methyl 4-(2,6-dimethoxyphenyl)-2,4-dioxobutanoate sodium salt (8a) and 1-naphthylhydrazine hydrochloride (9b) according to the general method (55%). ¹H NMR (CDCl₃) δ 7.67-7.83 (m, 3H), 7.38-7.49 (m, 2H), 7.22-7.33 (m, 2H), 7.05-7.17 (m, 2H), 6.34 (d, J=8.29 Hz, 2H), 3.96 (s, 3H), 3.41 (br. s., 6H).

### Methyl 5-(2-methoxyphenyl)-1-naphthalen-1-yl-1H-pyrazole-3-carboxylate (10h)

Ester 10h was prepared from methyl 4-(2-methoxyphenyl)-2,4-dioxobutanoate sodium salt (80 and 1-naphthylhydrazine hydrochloride (9b) according to the general method (40%). ¹H NMR (CDCl₃) δ 7.78-7.88 (m, 2H), 7.62-7.71 (m, 1H), 7.44-7.53 (m, 2H), 7.29-7.36 (m, 1H), 7.24-7.29 (m, 1H), 7.10-7.24 (m, 3H), 6.81 (dt, J=0.80, 7.51 Hz, 1H), 6.64 (d, J=8.19 Hz, 1H), 3.97 (s, 3H), 3.13 (s, 3H).

### Methyl 5-(2,6-dimethoxyphenyl)-1-(4-fluorophenyl)-1H-pyrazole-3-carboxylate (10i)

Ester 10i was prepared from methyl 4-(2,6-dimethoxyphenyl)-2,4-dioxobutanoate sodium salt (8a) and 4-fluorophenyl hydrazine hydrochloride (9c) according to the general method (55%). ¹H NMR (CDCl₃) δ 7.22-7.33 (m, 3H), 6.89-7.00 (m, 3H), 6.51 (d, J=8.29 Hz, 2H), 3.96 (s, 3H), 3.59 (s, 6H).

### Methyl 1-(4-fluorophenyl)-5-(2-methoxyphenyl)-1H-pyrazole-3-carboxylate (10j)

Ester 10j was prepared from methyl 4-(2-methoxyphenyl)-2,4-dioxobutanoate sodium salt (80 and 4-fluorophenyl hydrazine hydrochloride (9c) according to the general method (45%). ¹H NMR (CDCl₃) δ 7.32-7.43 (m, 1H), 7.23-7.32 (m, 3H), 6.92-7.04 (m, 3H), 6.81 (d, J=8.48 Hz, 2H), 3.97 (s, 3H), 3.44 (s, 3H).

### 1-(7-Chloroquinolin-4-yl)-5-(2,6-dimethoxyphenyl)-1H-pyrazole-3-carboxylic Acid (11a)

Pyrazole acid 11a was prepared from ester 10a according to the general methyl ester hydrolysis method (78%). ¹H NMR (300 MHz, DMSO-d₆) δ 8.90 (d, J=4.71 Hz, 1H), 8.17 (s, 1H), 7.73 (s, 2H), 7.26 (t, J=8.38 Hz, 1H), 7.20 (d, J=4.52 Hz, 1H), 6.99 (s, 1H), 6.54 (d, J=8.48 Hz, 2H), 3.39 (s, 6H).

### 1-(7-Chloroquinolin-4-yl)-5-(2,6-dimethoxyphenyl)-4-ethyl-1H-pyrazole-3-carboxylic Acid (11b)

Pyrazole acid 11b was prepared from ester 10b according to the general methyl ester hydrolysis method (80%). ¹H NMR (CDCl₃) δ 10.97 (br. s., 1H), 8.87 (d, J=4.8 Hz, 1H), 8.19 (d, J=1.9 Hz, 1H), 7.94 (d, J=9.0 Hz, 1H), 7.49 (dd, J=2.03, 9.09 Hz, 1H), 7.30-7.09 (m, 2H), 6.42 (d, J=8.4 Hz, 2H), 3.50 (s, 6H), 2.68 (q, J=7.4 Hz, 2H), 1.16 (t, J=7.4 Hz, 3H). MS (ESI) m/z: 436.5 (M−H+, 80%).

### 1-(7-Chloroquinolin-4-yl)-5-(2,5-dimethoxyphenyl)-1H-pyrazole-3-carboxylic Acid (11c)

Pyrazole acid 11c was prepared from ester 10c according to the general methyl ester hydrolysis method (94%). ¹H NMR (CDCl₃) δ 8.80 (d, 4.7 Hz, 1H), 8.18 (d, 1.9 Hz, 1H), 7.91 (d, 9.2 Hz, 1H), 7.56 (dd, 1.9, 9.0 Hz, 1H), 7.19 (s, 1H), 7.02 (d, 4.7 Hz, 1H), 6.91-6.82 (m, 2H), 6.57 (d, 9.0 Hz, 1H), 3.75 (s, 3H), 2.92 (m, 3H).

### 1-(7-Chloroquinolin-4-yl)-5-(2,4-dimethoxyphenyl)-1H-pyrazole-3-carboxylic Acid (11d)

Pyrazole acid 11d was prepared from ester 10d according to the general methyl ester hydrolysis method (78%). ¹H NMR (CDCl₃) δ 8.82 (d, J=4.7 Hz, 1H), 8.18 (d, J=2.0 Hz, 1H), 7.89 (d, J=9.0 Hz, 1H), 7.55 (dd, J=2.1, 9.0 Hz, 1H), 7.21 (d, J=8.4 Hz, 1H), 7.12 (s, 1H), 7.04 (d, J=4.7 Hz, 1H), 6.48 (dd, J=2.3, 8.5 Hz, 1H), 6.20 (d, J=2.2 Hz, 1H), 5.30 (s, 2H), 3.78 (s, 3H), 2.99 (s, 3H). or ¹H NMR (DMSO-d₆) δ 8.91 (d, J=4.7 Hz, 1H), 8.22 (d, J=1.7 Hz, 1H), 7.78 (s, 1H), 7.76 (d, J=1.98 Hz, 1H), 7.32 (d, J=8.4 Hz, 1H), 7.24 (d, J=4.7 Hz, 1H), 7.04 (s, 1H), 6.56 (dd, J=2.2, 8.5 Hz, 1H), 6.34 (d, J=2.2 Hz, 1H), 3.72 (s, 3H), 2.92 (s, 3H).

### 1-(7-chloroquinolin-4-yl)-5-(2,6-difluorophenyl)-1H-pyrazole-3-carboxylic acid (11e)

Pyrazole acid 11e was prepared from ester 10e according to the general methyl ester hydrolysis method (94%). ¹H NMR (CD₃OD) δ 8.9 (d, J=4.7 Hz, 1H), 8.1 (s, 1H), 7.8 (d, J=9.0 Hz, 1H), 7.62 (d, J=9.0 Hz, 1H), 7.23-7.35 (m, 1H), 7.04 (s, 1H), 6.54 (d, J=8.5 Hz, 2H).

### 1-(7-Chloroquinolin-4-yl)-5-(2-methoxyphenyl)-1H-pyrazole-3-carboxylic Acid (11f)

Pyrazole acid 11f was prepared from ester 10f according to the general methyl ester hydrolysis method (92%). ¹H NMR (DMSO-d₆) δ 8.80 (d, J=4.7 Hz, 1H), 8.15 (d, J=2.1 Hz, 1H), 7.98 (d, J=9.0 Hz, 1H), 7.69 (dd, J=2.1, 9.0 Hz, 1H), 7.25-7.39 (m, 2H), 7.04 (d, J=4.7 Hz, 1H), 6.97 (t, J=7.4 Hz, 1H), 6.79 (d, J=8.1 Hz, 1H), 6.70 (s, 1H), 2.91 (s, 3H).

### 5-(2,6-Dimethoxyphenyl)-1-naphthalen-1-yl-1H-pyrazole-3-carboxylic Acid (11g)

Pyrazole acid 11g was prepared from ester 10g according to the general methyl ester hydrolysis method (70%). ¹H NMR (CDCl₃) δ 7.78-7.86 (m, 2H), 7.72 (dd, J=3.5, 6.3 Hz, 1H), 7.44-7.52 (m, 2H), 7.22-7.35 (m, 2H), 7.10-7.19 (m, 2H), 6.35 (d, J=8.3 Hz, 2H), 3.41 (s, 6H).

### 5-(2-Methoxyphenyl)-1-naphthalen-1-yl-1H-pyrazole-3-carboxylic Acid (11h)

Pyrazole acid 11h was prepared from ester 11h according to the general methyl ester hydrolysis method (92%). ¹H NMR (CDCl₃) δ 7.81-7.91 (m, 2H), 7.68 (dd, J=3.4, 6.2 Hz, 1H), 7.46-7.56 (m, 2H), 7.28-7.37 (m, 1H), 7.14-7.24 (m, 3H), 6.83 (t, J=7.4 Hz, 1H), 6.65 (d, J=8.3 Hz, 1H), 3.12 (s, 3H). MS (ESI) m/z: 343.2 (M−H⁺).

### 5-(2,6-Dimethoxyphenyl)-1-(4-fluorophenyl)-1H-pyrazole-3-carboxylic Acid (11i)

Pyrazole acid 11i was prepared from ester 10i according to the general methyl ester hydrolysis method (93%). ¹H NMR (CDCl₃) δ 7.23-7.36 (m, 3H), 6.91-7.03 (m, 3H), 6.52 (d, J=8.48 Hz, 2H), 3.60 (s, 6H).

### 1-(4-Fluorophenyl)-5-(2-methoxyphenyl)-1H-pyrazole-3-carboxylic Acid (11j)

Pyrazole acid 11j was prepared from ester 10j according to the general methyl ester hydrolysis method (91%). ¹H NMR (CDCl₃) δ 7.34-7.44 (m, 1H), 7.22-7.33 (m, 3H), 6.95-7.07 (m, 4H), 6.82 (d, J=8.29 Hz, 1H), 3.44 (s, 3H).

### 1-(7-chloroquinolin-4-yl)-5-(2,6-dimethoxyphenyl)-N-tricyclo[3.3.1.13,7]dec-2-yl-1H-pyrazole-3-carboxamide (13)

Compound 13 was prepared from 1-(7-chloroquinolin-4-yl)-5-(2,6-dimethoxyphenyl)-1H-pyrazole-3-carboxylic acid (11a) and 2-aminoadamantane hydrochloride (12e) following the general amide coupling method (off-white powder, 65%).¹H NMR δ (CDCl₃) 8.77 (d, J=4.71 Hz, 1H), 8.16 (d, J=1.88 Hz, 1H), 7.96 (d, J=9.04 Hz, 1H), 7.55 (dd, J=1.88, 9.04 Hz, 1H), 7.24-7.37 (m, 2H), 7.13 (s, 1H), 6.92-7.02 (m, 2H), 6.64 (d, J=8.29 Hz, 1H), 4.28 (d, J=8.10 Hz, 1H), 2.98 (s, 6H), 2.02-2.11 (m, 2H), 1.80-1.95 (m, 9H), 1.7-1.79 (m, 2H), 1.59-1.70 (m, 2H). Anal. (C₃₁H₃₁ClN₄O₃) C, H, N.

### tert-Butyl (2S)-({[1-(7-Chloroquinolin-4-yl)-5-(2,6-dimethoxyphenyl)-1H-pyrazol-3-yl]carbonyl}amino) (cyclohexyl)ethanoate (14a)

Following the general amide coupling procedures, 14a was obtained from 1-(7-chloroquinolin-4-yl)-5-(2,6-dimethoxyphenyl)-1H-pyrazole-3-carboxylic acid (11a) and tert-butyl (2S)-amino(cyclohexyl)ethanoate hydrochloride (12d). The material was purified by flash chromatography (0-100% EtOAc/hexanes) to afford 14a (84%) as a pale yellow film. ¹H NMR (CDCl₃) δ 8.78 (d, J=4.71 Hz, 1H), 8.12 (d, J=1.98 Hz, 1H), 7.97 (d, J=9.04 Hz, 1H), 7.50 (dd, J=2.07, 9.04 Hz, 1H), 7.42 (d, J=9.04 Hz, 1H), 7.21 (t, J=8.43 Hz, 1H), 7.01-7.12 (m, 2H), 6.39 (d, J=8.19 Hz, 2H), 4.66 (dd, J=5.13, 9.09 Hz, 1H), 3.40 (br. s., 6H), 1.54-1.99 (m, 8H), 1.44-1.53 (m, 10H), 1.01-1.32 (m, 7H).

### (2S)-({[1-(7-Chloroquinolin-4-yl)-5-(2,6-dimethoxyphenyl)-1H-pyrazol-3-yl]carbonyl}amino)-(cyclohexyl)ethanoic Acid (14b)

Following the general tert-butyl ester hydrolysis procedure 14b was obtained as a pale yellow solid in 91% yield from 14a. ¹H NMR (CDCl₃) δ 9.10 (br. s., 1H), 8.74 (br. s., 1H), 8.31 (d, J=8.9 Hz, 1H), 7.78 (d, J=8.9 Hz, 1H), 7.64-7.41 (m, 2H), 7.39-7.25 (m, 1H), 6.48 (d, J=7.5 Hz, 2H), 4.80 (dd, J=4.7, 7.9 Hz, 1H), 3.47 (br. s., 6H), 2.10-1.92 (m, 1H), 1.90-1.60 (m, 5H), 1.32-1.09 (m, 5H). Anal. (C₂₉H₂₉ClN₄O₅) C, H, N.

### tert-Butyl (2S)-({[1-(7-Chloroquinolin-4-yl)-5-(2,5-dimethoxyphenyl)-1H-pyrazol-3-yl]carbonyl}-amino)(cyclohexyl)ethanoate (15a)

Following the general amide coupling procedure, 15a was obtained from 1-(7-chloroquinolin-4-yl)-5-(2,5-dimethoxyphenyl)-1H-pyrazole-3-carboxylic acid (11c) and tert-butyl (2S)-amino(cyclohexyl)ethanoate hydrochloride (12d) as a pale yellow film (70%). ¹H NMR (CDCl₃) δ 8.78 (d, 4.7 Hz, 1H), 8.16 (d, 1.9 Hz, 1H), 8.01 (d, 9.2 Hz, 1H), 7.55 (dd, 2.3, 9.0 Hz, 1H), 7.40 (d, 9.0 Hz, 1H), 7.14-7.11 (m, 1H), 7.00 (d, 4.7 Hz, 1H), 6.90 (d, 3.0 Hz, 1H), 6.83 (dd, 3.2, 9.0 Hz, 1H), 6.55 (d, 9.0 Hz, 1H), 4.69-4.62 (m, 1H), 3.72 (s, 3H), 2.89 (m, 3H), 2.05-1.60 (m, 6H), 1.50 (s, 9H), 1.39-1.11 (m, 5H). MS (ESI) m/z: 494.5 (M−H⁺).

### (2S)-({[1-(7-chloroquinolin-4-yl)-5-(2,5-dimethoxyphenyl)-1H-pyrazol-3-yl]carbonyl}amino) (cyclohexyl)ethanoic acid (15b)

Following the general tert-butyl ester hydrolysis procedure 15b was obtained (92%) as a white foam from 15a. ¹H NMR (CDCl₃) δ 9.0 (d, J=5.3 Hz, 1H), 8.39 (d, J=1.8 Hz, 1H), 8.17 (d, J=9.2 Hz, 1H), 7.7 (dd, J=1.9, 9.0 Hz, 1H), 7.3 (d, J=9.0 Hz, 1H), 7.27-7.16 (m, 2H), 6.9 (d, J=3.0 Hz, 1H), 6.83 (dd, J=3.2, 9.0 Hz, 1H), 6.58 (d, J=9.0 Hz, 1H), 4.81-4.72 (m, 1H), 3.78 (s, 3H), 2.9 (m, 3H), 2.05-1.60 (m, 6H), 1.37-1.03 (m, 5H). MS (ESI) m/z: 547.8 (M−H⁺). Anal. (C₂₉H₂₉ClN₄O₅) C, H, N.

### tert-Butyl (2S)-({[1-(7-Chloroquinolin-4-yl)-5-(2,4-dimethoxyphenyl)-1H-pyrazol-3-yl]carbonyl}-amino) (cyclohexyl)ethanoate (16a)

Following the general amide coupling procedure, 16a was obtained as a pale yellow film (86%) from 1-(7-chloroquinolin-4-yl)-5-(2,4-dimethoxyphenyl)-1H-pyrazole-3-carboxylic acid (11d) and tert-butyl (2S)-amino(cyclohexyl)ethanoate hydrochloride (12d). ¹H NMR (CDCl₃) δ 8.79 (d, J=4.71 Hz, 1H), 8.16 (d, J=1.98 Hz, 1H), 7.96 (d, J=9.04 Hz, 1H), 7.54 (dd, J=2.07, 9.04 Hz, 1H), 7.40 (d, J=9.04 Hz, 1H), 7.21 (d, J=8.38 Hz, 1H), 7.07 (s, 1H), 7.01 (d, J=4.62 Hz, 1H), 6.47 (dd, J=2.26, 8.48 Hz, 1H), 6.18 (d, J=2.26 Hz, 1H), 4.66 (dd, J=5.13, 9.09 Hz, 1H), 3.77 (s, 3H), 2.96 (s, 3H), 1.56-1.81 (m, 11H), 1.48 (d, J=4.52 Hz, 16H), 1.00-1.34 (m, 10H).

### (2S)-({[1-(7-Chloroquinolin-4-yl)-5-(2,4-dimethoxyphenyl)-1H-pyrazol-3-yl]carbonyl}-amino)-(cyclohexyl)ethanoic Acid (16b)

Following the general tert-butyl ester hydrolysis procedure 16b was obtained as a yellow solid in 86% yield from 16a. ¹H NMR (CDCl₃) δ 9.11 (br. s., 1H), 8.48 (s, 1H), 8.27 (d, J=9.1 Hz, 1H), 7.80 (d, J=8.9 Hz, 1H), 7.46 (d, J=8.6 Hz, 1H), 7.42-7.35 (m, 1H), 7.31 (d, J=8.5 Hz, 1H), 7.26 (s, 1H), 7.12 (s, 1H), 6.59 (d, J=8.4 Hz, 1H), 6.23 (d, J=1.7 Hz, 1H), 4.73 (dd, J=5.4, 8.3 Hz, 1H), 3.81 (s, 3H), 3.02 (s, 3H), 1.87-1.61 (m, 4H), 1.37-1.04 (m, 6H). Anal. (C₂₉H₂₉ClN₄O₅) C, H, N.

### tert-Butyl (2S)-({[1-(7-Chloroquinolin-4-yl)-5-(2-methoxyphenyl)-1H-pyrazol-3-yl]carbonyl}-amino) (cyclohexyl)ethanoate (17a)

Following the general amide coupling procedure, 17a was obtained from 1-(7-chloroquinolin-4-yl)-5-(2-methoxyphenyl)-1H-pyrazole-3-carboxylic acid (110 and tert-butyl (2S)-amino(cyclohexyl)ethanoate hydrochloride (12d) in 94% yield as a pale yellow solid. ¹H NMR (CDCl₃) δ 8.77 (d, J=4.71 Hz, 1H), 8.16 (d, J=1.88 Hz, 1H), 7.98 (d, J=9.04 Hz, 1H), 7.55 (dd, J=2.02, 9.09 Hz, 1H), 7.41 (d, J=9.14 Hz, 1H), 7.23-7.35 (m, 2H), 7.09-7.16 (m, 1H), 6.89-7.03 (m, 2H), 6.64 (d, J=8.10 Hz, 1H), 4.66 (dd, J=5.04, 9.09 Hz, 1H), 2.98 (s, 3H), 1.53-1.99 (m, 7H), 1.49 (s, 9H), 1.01-1.35 (m, 6H).

### (2S)-({[1-(7-chloroquinolin-4-yl)-5-(2-methoxyphenyl)-1H-pyrazol-3-yl]carbonyl}amino)(cyclohexyl) ethanoic acid (17b)

Following the general tert-butyl ester hydrolysis procedure 17b was obtained as a pale yellow film in 74% yield from 17a. ¹H NMR (CDCl₃) δ 9.09 (br. s., 1H), 8.76 (br. s., 1H), 8.33 (d, J=8.9 Hz, 1H), 7.82 (d, J=8.9 Hz, 1H), 7.57-7.32 (m, 4H), 7.22 (s, 1H), 7.10 (t, J=6.9 Hz, 1H), 6.70 (d, J=7.7 Hz, 1H), 4.78 (dd, J=4.8, 8.2 Hz, 1H), 3.04 (br. s., 3H), 2.10-1.90 (m, 1H), 1.89-1.60 (m, 5H), 1.25-1.07 (m, 5H). Anal. (C₂₈H₂₇ClN₄O₄) C, H, N. ***MS?

### tert-Butyl (2S)-({[1-(7-Chloroquinolin-4-yl)-5-(2,6-difluorophenyl)-1H-pyrazol-3-yl]carbonyl}-amino)(cyclohexyl)ethanoate (18a)

Following the general amide coupling procedure, 18a was obtained from 1-(7-chloroquinolin-4-yl)-5-(2,6-difluorophenyl)-1H-pyrazole-3-carboxylic acid (11e) and tert-butyl (2S)-amino(cyclohexyl)ethanoate hydrochloride (12d). ¹H NMR (CDCl₃) δ 8.89 (d, 4.7 Hz, 1H), 8.15 (d, 4.7 Hz, 1H), 7.75 (d, 9.0 Hz, 1H), 7.75 (d, 9.0 Hz, 1H), 7.52 (dd, 1.9, 9.0 Hz, 1H), 7.4 (d, 9.0 Hz, 1H), 7.32-7.25 (m, 3H), 7.2 (d, 9.0 Hz, 1H), 6.8 (t, 7.6 Hz, 1H), 4.69-4.63 (m, 1H), 1.9-1.85 (m, 1H), 1.84-1.60 (m, 5H), 1.5 (s, 9H), 1.32-1.06 (m, 5H).

### (2S)-({[1-(7-Chloroquinolin-4-yl)-5-(2,6-difluorophenyl)-1H-pyrazol-3-yl]carbonyl}amino) (cyclohexyl)ethanoic Acid (18b)

Following the general tert-butyl ester hydrolysis procedure, 18b was obtained as a white foam (90%) from 18a. ¹H NMR (CDCl₃) δ 8.91 (d, J=4.7 Hz, 1H), 8.18 (s, 1H), 8.17 (d, J=9.2 Hz, 1H), 7.74 (d, J=9.0 Hz, 1H), 7.53 (dd, J=1.9, J=9.0 Hz, 1H), 7.4 (d, J=9.0 Hz, 1H), 7.20-7.05 (m, 4H), 6.8 (t, J=7.6 Hz, 1H), 4.82-4.74 (m, 1H), 2.08-1.94 (m, 1H), 1.89-1.60 (m, 5H), 1.37-1.05 (m, 5H). Anal. (C₂₇H₂₃ClF₂N₄O₃) C, H, N.

### tert-Butyl (2S)-({[1-(7-Chloroquinolin-4-yl)-5-(2,6-dimethoxyphenyl)-4-ethyl-1H-pyrazol-3-yl]carbonyl}amino)(cyclohexyl)ethanoate (19a)

Following the general amide coupling procedures, 19a was obtained as a pale yellow film (86%) from 1-(7-chloroquinolin-4-yl)-5-(2,6-dimethoxyphenyl)-4-ethyl-1H-pyrazole-3-carboxylic acid (11b) and tert-butyl (2S)-amino(cyclohexyl)ethanoate hydrochloride (12d). ¹H NMR (CDCl₃) δ 8.76 (d, J=4.62 Hz, 1H), 8.09 (d, J=2.07 Hz, 1H), 7.98 (d, J=9.14 Hz, 1H), 7.48 (td, J=2.19, 9.09 Hz, 2H), 7.21 (t, J=8.43 Hz, 1H), 7.09 (d, J=4.62 Hz, 1H), 6.40 (dd, J=8.43, 15.59 Hz, 2H), 4.64 (dd, J=4.94, 9.00 Hz, 1H), 3.53 (s, 3H), 3.42 (s, 3H), 2.54-2.82 (m, 2H), 1.61-1.95 (m, 6H), 1.49 (s, 9H), 1.17-1.36 (m, 5H), 1.06-1.15 (m, 3H). MS (ESI) m/z: 633.7 (M+H⁺).

### (2S)-({[1-(7-Chloroquinolin-4-yl)-5-(2,6-dimethoxyphenyl)-4-ethyl-1H-pyrazol-3-yl]carbonyl}amino)(cyclohexyl)ethanoic Acid (19b)

Following the general tert-butyl ester hydrolysis procedure 19b was obtained as a pale yellow solid in 96% yield from 19a. ¹H NMR (CDCl₃) δ 9.12 (d, J=5.7 Hz, 1H), 8.43 (s, 1H), 8.38 (d, J=9.2 Hz, 1H), 7.77 (dd, J=1.3, 9.3 Hz, 1H), 7.56 (d, J=8.7 Hz, 1H), 7.48 (d, J=5.7 Hz, 1H), 7.34 (t, J=8.4 Hz, 1H), 6.51 (dd, J=8.5, 15.5 Hz, 2H), 4.77 (dd, J=5.2, 8.7 Hz, 1H), 3.67-3.45 (m, 6H), 2.66 (dd, J=3.2, 7.3 Hz, 2H), 2.12-1.91 (m, 1H), 1.90-1.60 (m, 6H), 1.38-1.16 (m, 4H), 1.15-1.01 (m, 3H). MS (ESI) m/z: 575.8 (M−H⁺). Anal. (C₃₁H₃₃ClN₄O₅) C, H, N.

### Methyl 1-({[1-(7-Chloroquinolin-4-yl)-5-(2,6-dimethoxyphenyl)-1H-pyrazol-3-yl]carbonyl}amino) cycloheptanecarboxylate (20a)

Following the general amide coupling procedure, 20a was obtained from 1-(7-chloroquinolin-4-yl)-5-(2,6-dimethoxyphenyl)-1H-pyrazole-3-carboxylic acid (11a) and methyl 1-aminocycloheptanecarboxylate hydrochloride (12c) as an off-white powder in 65% yield. ¹H NMR (CDCl₃) δ 8.77 (d, J=4.71 Hz, 1H), 8.16 (d, J=1.88 Hz, 1H), 7.96 (d, J=9.04 Hz, 1H), 7.55 (dd, J=1.88, 9.04 Hz, 1H), 7.24-7.37 (m, 2H), 7.13 (s, 1H), 6.92-7.02 (m, 2H), 6.64 (d, J=8.29 Hz, 1H), 4.28 (d, J=8.10 Hz, 1H), 2.98 (s, 6H), 2.02-2.11 (m, 2H), 1.80-1.95 (m, 9H), 1.7-1.79 (m, 2H), 1.59-1.70 (m, 2H).

### 1-({[1-(7-Chloroquinolin-4-yl)-5-(2,6-dimethoxyphenyl)-1H-pyrazol-3-yl]carbonyl}amino)cyclo-heptanecarboxylic Acid (20b)

Following the general methyl ester hydrolysis procedure, 20b was obtained from 20a as a white powder in 86% yield. ¹H NMR (CDCl₃) δ 8.80 (d, 4.7 Hz, 1H), 8.14 (d, 2.1 Hz, 1H), 7.90 (d, 9.0 Hz, 1H), 7.50 (dd, 2.1, 9.0 Hz, 1H), 7.24-7.17 (m, 1H), 7.11 (s, 1H), 7.07 (d, 4.7 Hz, 1H) 6.40 (d, 8.7 Hz, 2H), 3.42 (s, 6H), 2.42-2.28 (m, 2H), 2.24-2.13 (m, 2H), 1.74-1.53 (m, 10H). Anal. (C₂₉H₂₉ClN₄O₅) C, H, N.

### Methyl 1-({[1-(7-Chloroquinolin-4-yl)-5-(2,6-dimethoxyphenyl)-1H-pyrazol-3-yl]carbonyl}amino) cyclohexanecarboxylate (21a)

Following the general amide coupling procedures, 21a was obtained from 1-(7-chloroquinolin-4-yl)-5-(2,6-dimethoxyphenyl)-1H-pyrazole-3-carboxylic acid (11a) and ethyl 1-aminocyclohexanecarboxylate hydrochloride (12b) as a white solid in 87% yield. 8.74-8.84 (m, 1H), 8.14 (d, J=2.07 Hz, 1H), 7.96 (d, J=9.04 Hz, 1H), 7.52 (dd, J=2.07, 9.04 Hz, 1H), 7.15-7.25 (m, 1H), 7.04-7.15 (m, 2H), 6.39 (d, J=8.48 Hz, 2H), 4.25 (q, J=7.16 Hz, 2H), 3.41 (s, 6H), 2.18 (d, J=13.56 Hz, 2H), 1.86-2.02 (m, 2H), 1.43-1.74 (m, 6H), 1.23-1.34 (m, 3H).

### 1-({[1-(7-Chloroquinolin-4-yl)-5-(2,6-dimethoxyphenyl)-1H-pyrazol-3-yl]carbonyl}amino)cyclo-hexanecarboxylic Acid (21b)

Following the general methyl ester hydrolysis procedure, 21b was obtained from 21a in 87% yield as a white solid. ¹H NMR (DMSO-d6) δ 8.92 (d, J=4.7 Hz, 1H), 8.15 (d, J=2.1 Hz, 1H), 7.86-7.75 (m, 2H), 7.69 (dd, J=2.1, 9.0 Hz, 1H), 7.30-7.21 (m, 2H), 6.95 (s, 1H), 6.53 (d, J=8.7 Hz, 1H), 3.43 (s, 6H), 2.17-2.06 (m, 2H), 1.85-1.72 (m, 2H), 1.62-1.25 (m, 6H). MS (ESI) m/z: 547.8 (M−H⁺). Anal. (C₂₈H₂₇ClN₄O₅) C, H, N.

### Methyl 1-({[1-(7-Chloroquinolin-4-yl)-5-(2,6-dimethoxyphenyl)-1H-pyrazol-3-yl]carbonyl}amino) cyclopentanecarboxylate (22a)

Following the general amide coupling procedures, 22a was obtained from 1-(7-chloroquinolin-4-yl)-5-(2,6-dimethoxyphenyl)-1H-pyrazole-3-carboxylic acid (11a) and methyl 1-aminocyclopentanecarboxylate hydrochloride (12a) as a white powder in 85% yield. ¹H NMR (CDCl₃) δ 8.78 (d, J=4.71 Hz, 1H), 8.12 (d, J=2.07 Hz, 1H), 7.94 (d, J=9.04 Hz, 1H), 7.52 (dd, J=2.07, 9.04 Hz, 1H), 7.29-7.22 (m, 1H), 7.21-7.16 (m, 1H), 7.11-7.05 (m, 1H), 6.39 (d, J=8.48 Hz, 2H), 3.78 (s, 3H), 3.41 (s, 6H), 2.42-2.28 (m, 2H), 2.17-2.05 (m, 2H), 1.90-1.80 (m, 4H).

### 1-({[1-(7-Chloroquinolin-4-yl)-5-(2,6-dimethoxyphenyl)-1H-pyrazol-3-yl]carbonyl}amino)cyclo-pentanecarboxylic Acid (22b)

Following the general methyl ester hydrolysis procedure, 22b was obtained from 22a as a white powder in 72% yield. ¹H NMR (CDCl₃) δ 8.80 (d, J=4.7 Hz, 1H), 8.14 (d, J=2.1 Hz, 1H), 7.90 (d, J=9.0 Hz, 1H), 7.53 (dd, J=2.1, J=9.0 Hz, 1H), 7.30-7.18 (m, 1H), 7.11 (s, 1H), 7.07 (d, J=4.7 Hz, 1H) 6.40 (d, J=8.7 Hz, 2H), 3.42 (s, 6H), 2.54-2.40 (m, 2H), 2.20-2.07 (m, 2H), 1.90-1.77 (m, 4H). Anal. (C₂₈H₂₇ClN₄O₅) C, H, N.

### Methyl 1-({[1-(7-Chloroquinolin-4-yl)-5-(2-methoxyphenyl)-1H-pyrazol-3-yl]carbonyl}amino) cyclohexanecarboxylate (23a)

Following the general amide coupling procedure, 23a was obtained from 1-(7-chloroquinolin-4-yl)-5-(2-methoxyphenyl)-1H-pyrazole-3-carboxylic acid (11f) and ethyl 1-aminocyclo-hexanecarboxylate hydrochloride (12b) as a white solid in 63% yield. ¹H NMR (CDCl₃) δ 8.78 (d, J=4.71 Hz, 1H), 8.17 (d, J=1.88 Hz, 1H), 7.96 (d, J=9.04 Hz, 1H), 7.56 (dd, J=2.17, 9.14 Hz, 1H), 7.24-7.34 (m, 2H), 7.10 (s, 1H), 6.90-7.01 (m, 1H), 6.64 (d, J=8.67 Hz, 2H), 3.78 (s, 3H), 2.99 (s, 3H), 2.18 (d, J=13.75 Hz, 2H), 1.88-2.01 (m, 2H), 1.63-1.74 (m, 6H).

### 1-({[1-(7-Chloroquinolin-4-yl)-5-(2-methoxyphenyl)-1H-pyrazol-3-yl]carbonyl}amino)cyclohexane carboxylic Acid (23b)

Following the general methyl ester hydrolysis procedure, 23b was obtained from 23a as a white solid in 77% yield. ¹H NMR (CDCl₃) δ 8.8 (d, J=4.7 Hz, 1H), 8.17 (d, J=2.1 Hz, 1H), 7.9 (d, J=9.0 Hz, 1H), (m, 2H), 7.5 (dd, J=2.1, J=9.0 Hz, 1H), 7.35-7.21 (m, 1H), 7.2-7.11 (m, 2H), 6.63 (d, J=8.7 Hz, 1H), 2.98 (s, 3H), 2.30-2.16 (m, 2H), 2.10-1.96 (m, 2H), 1.76-1.53 (m, 6H). Anal. (C₂₈H₂₇ClN₄O₄) C, H, N.

### tert-Butyl (2S)-Cyclohexyl({[5-(2,6-dimethoxyphenyl)-1-naphthalen-1-yl-1H-pyrazol-3-yl]carbonyl}amino)ethanoate (24a)

Following the general amide coupling procedures, 24a was obtained from 5-(2,6-dimethoxyphenyl)-1-naphthalen-1-yl-1H-pyrazole-3-carboxylic acid (11g) and tert-butyl (2S)-amino(cyclohexyl)ethanoate hydrochloride (12d) as a white solid in 53% yield. ¹H NMR (CDCl₃) δ 7.88-7.72 (m, 3H), 7.51-7.38 (m, 3H), 7.36-7.21 (m, 2H), 7.11 (t, J=8.4 Hz, 1H), 7.07-7.03 (m, 1H), 6.33 (d, J=5.9 Hz, 2H), 4.66 (dd, J=5.4, 9.1 Hz, 1H), 3.40 (br. s., 6H), 1.96-1.52 (m, 8H), 1.47 (s, 9H), 1.33-1.02 (m, 6H).

### (2S)-Cyclohexyl({[5-(2,6-dimethoxyphenyl)-1-naphthalen-1-yl-1H-pyrazol-3-yl]carbonyl}amino)-ethanoic Acid (24b)

Following the general tert-butyl ester hydrolysis procedure 24b was obtained as a slightly yellow solid in 53% yield from 24a. ¹H NMR (CDCl₃) δ 7.82 (d, J=6.8 Hz, 2H), 7.73 (d, J=6.4 Hz, 2H), 7.48 (dd, J=3.2, 6.2 Hz, 2H), 7.38-7.21 (m, 2H), 7.19-7.01 (m, 2H), 6.33 (d, J=8.1 Hz, 2H), 4.73 (dd, J=5.8, 8.3 Hz, 1H), 3.40 (br. s., 6H), 1.93 (br. s., 1H), 1.83-1.55 (m, 5H), 1.23-0.99 (m, 5H). Anal. (C₃₀H₃₁N₃O₅) C, H, N.

### tert-Butyl (2S)-Cyclohexyl({[5-(2-methoxyphenyl)-1-naphthalen-1-yl-1H-pyrazol-3-yl]carbonyl}-amino)ethanoate (25a)

Following the general amide coupling procedure, 25a was obtained from 5-(2-methoxyphenyl)-1-naphthalen-1-yl-1H-pyrazole-3-carboxylic acid (11h) and tert-butyl (2S)-amino-(cyclohexyl)ethanoate hydrochloride (12d) in 81% yield as a colorless film. ¹H NMR (CDCl₃) δ 7.84 (s, 1H), 7.81 (s, 1H), 7.75 (d, J=3.49 Hz, 1H), 7.55-7.46 (m, 2H), 7.43 (d, J=9.14 Hz, 1H), 7.36-7.28 (m, 1H), 7.23-7.14 (m, 3H), 7.12 (s, 1H), 6.85-6.75 (m, 1H), 6.61 (d, J=8.57 Hz, 1H), 4.66 (dd, J=5.37, 9.14 Hz, 1H), 3.07 (s, 3H), 1.96-1.57 (m, 7H), 1.47 (s, 9H), 1.32-1.05 (m, 6H).

### (2S)-Cyclohexyl({[5-(2-methoxyphenyl)-1-naphthalen-1-yl-1H-pyrazol-3-yl]carbonyl}amino) ethanoic Acid (24b)

Following the general tert-butyl ester hydrolysis procedure 24b was obtained from 24a as a white foam (43%). ¹H NMR (CDCl₃) δ 7.91-7.81 (m, 2H), 7.73-7.61 (m, 2H), 7.56-7.47 (m, 2H), 7.39-7.29 (m, 1H), 7.25-7.18 (m, 2H), 7.15 (s, 2H), 6.80 (t, J=7.4 Hz, 1H), 6.63 (d, J=8.3 Hz, 1H), 4.74 (dd, J=5.7, 8.8 Hz, 1H), 3.12 (s, 3H), 1.93 (br. s., 1H), 1.83-1.53 (m, 5H), 1.24-1.02 (m, 5H). Anal. (C₂₉H₂₉N₃O₄) C, H, N.

### Methyl 1-({[5-(2,6-Dimethoxyphenyl)-1-naphthalen-1-yl-1H-pyrazol-3-yl]carbonyl}amino)cyclo-hexane carboxylate (26a)

Following the general amide coupling procedure, 26a was obtained from 5-(2,6-dimethoxyphenyl)-1-naphthalen-1-yl-1H-pyrazole-3-carboxylic acid (11g) and ethyl 1-aminocyclohexane-carboxylate hydrochloride (12b) as a white solid in 98% yield. ¹H NMR (CDCl₃) δ 7.73-7.89 (m, 3H), 7.45-7.52 (m, 2H), 7.27-7.37 (m, 2H), 7.06-7.16 (m, 2H), 7.02 (s, 1H), 6.32 (d, J=8.3 Hz, 2H), 3.78 (s, 3H), 3.23-3.63 (m, 6H), 2.08-2.32 (m, 4H), 1.56 (br. s., 8H).

### 1-({[5-(2,6-Dimethoxyphenyl)-1-naphthalen-1-yl-1H-pyrazol-3-yl]carbonyl}amino)cyclohexane-carboxylic Acid (26b)

Following the general ethyl ester hydrolysis procedure, 26b was obtained from 26a as an off-white powder in 35% yield. ¹H NMR (CDCl₃) δ 7.88-7.78 (m, 2H), 7.77-7.67 (m, 1H), 7.54-7.45 (m, 2H), 7.37-7.29 (m, 1H), 7.28-7.22 (m, 4H), 7.15 (t, J=8.4 Hz, 1H), 7.08 (s, 1H), 6.34 (d, J=8.5 Hz, 2H), 3.41 (br. s., 6H), 2.24 (d, J=14.1 Hz, 2H), 2.10-1.94 (m, 2H), 1.79-1.48 (m, 6H). Anal. (C₃₀H₃₁N₃O₅) C, H, N.

### Methyl 1-({[5-(2-methoxyphenyl)-1-naphthalen-1-yl-1H-pyrazol-3-yl]carbonyl}amino)cyclohexane carboxylate (27a)

Following the general amide coupling procedures, 27a was obtained from 5-(2-methoxyphenyl)-1-naphthalen-1-yl-1H-pyrazole-3-carboxylic acid (11h) and ethyl 1-aminocyclohexane-carboxylate hydrochloride (12b) as a colorless film in 73% yield. ¹H NMR (CDCl₃) δ 7.93-7.80 (m, 2H), 7.80-7.71 (m, 1H), 7.59-7.46 (m, 2H), 7.40-7.29 (m, 1H), 7.25-7.12 (m, 4H), 7.10 (s, 1H), 6.87-6.74 (m, 1H), 6.62 (d, J=8.2 Hz, 1H), 3.77 (s, 3H), 3.09 (s, 3H), 2.13 (br. s., 2H), 1.96 (br. s., 3H), 1.71-1.30 (m, 10H).

### 1-({[5-(2-Methoxyphenyl)-1-naphthalen-1-yl-1H-pyrazol-3-yl]carbonyl}amino)cyclohexane-carboxylic Acid (27b)

Following the general ethyl ester hydrolysis procedure, 27b was obtained from 27a as a white foam (66%). ¹H NMR (CDCl₃) δ 7.92-7.82 (m, 2H), 7.68 (d, J=3.4 Hz, 1H), 7.53 (dd, J=3.3, 6.3 Hz, 2H), 7.38-7.30 (m, 1H), 7.25-7.09 (m, 5H), 6.82 (t, J=7.4 Hz, 1H), 6.63 (d, J=8.2 Hz, 1H), 3.10 (s, 3H), 2.22 (d, J=13.9 Hz, 2H), 2.01 (t, J=11.2 Hz, 2H), 1.77-1.19 (m, 8H), 0.97 (br. s., 1H). Anal. (C₂₈H₂₇N₃O₄) C, H, N.

### 2-({[5-(2,6-Dimethoxyphenyl)-1-(4-fluorophenyl)-1H-pyrazol-3-yl]carbonyl}amino) tricyclo[3.3.1.13,7]decane-2-carboxylic Acid (30)

Following the method of Quéré⁴⁷ 30 was obtained from 5-(2,6-dimethoxyphenyl)-1-(4-fluorophenyl)-1H-pyrazole-3-carboxylic acid (11i) and 9-aminobicyclo[3.3.1]nonane-9-carboxylic acid (120. Briefly, 11i was heated under reflux in SOCl₂ for 2 hr followed by cooling and concentration on a rotavap. The residue was then dissolved in toluene and evaporated three times to remove HCl. The residue was then taken up in dry THF and added to a well agitated mixture of 5:1 THF:water (15 mL/g of acid chloride) containing 1.1 eq 2-aminoadamantane-carboxylic acid and 2.2 eq of NaOH at 5° C. Following the addition, the stiffing was continued for 18 hr at ambient temp. After this time, the mixture is made acidic with 1 N HCl and extracted three times with CH₂Cl₂, dried over sodium sulfate and evaporated. The residue was chromatographed using a 0-15% CH₂Cl₂:MeOH gradient. Evaporation provided the desired compound as an off-white solid (71%). ¹H NMR (CDCl₃) δ 7.20-7.36 (m, 4H), 6.90-7.04 (m, 3H), 6.51 (d, J=8.48 Hz, 2H), 3.54-3.63 (m, 6H), 2.71-2.81 (m, 2H), 2.23 (d, J=12.81 Hz, 2H), 2.07 (d, J=12.24 Hz, 2H), 1.68-1.95 (m, 8H). MS (ESI) m/z: 518.9 (M−H⁺). Anal. (C₂₉H₃₀FN₃O₅) C, H, N.

### 2-({[1-(4-fluorophenyl)-5-(2-methoxyphenyl)-1H-pyrazol-3-yl]carbonyl}amino)tricyclo[3.3.1.13,7]decane-2-carboxylic acid (31)

Compound 31 was obtained using the same method as described for 30 starting from 5-(2-methoxyphenyl)-1-(4-fluorophenyl)-1H-pyrazole-3-carboxylic acid (11j) and 9-aminobicyclo[3.3.1]nonane-9-carboxylic acid (120. The crude compound was chromatographed on a 12 g silica gel column using a 0-30% gradient of (chloroform:methanol:ammonia 80:19:1) in CH₂Cl₂ on an ISCO Combiflash instrument. The first major peak observed was the desired product which gave a colorless film upon concentration (70.3 mg, 25%). The ESI mass spec shows a peak at 490.5 in the positive mode and 488.6 in the negative mode, which is consistent with the desired product. 1H NMR: (300 MHz, CHLOROFORM-d) δ 8.64 (d, J=4.24 Hz, 2H), 7.67-7.76 (m, 1H), 7.18-7.42 (m, 4H), 6.92-7.07 (m, 3H), 6.81 (d, J=8.19 Hz, 1H), 3.43 (s, 3H), 2.77 (br. s., 2H), 2.25 (d, J=12.72 Hz, 2H), 2.01-2.14 (m, 2H), 1.86 (d, J=7.06 Hz, 3H), 1.67-1.82 (m, 5H); ¹³C NMR: ¹³C NMR (75 MHz, CHLOROFORM-d) 164.1, 163.5, 160.2, 156.4, 149.0, 145.5, 143.2, 142.6, 142.4, 136.7, 136.5, 136.5, 131.2, 131.2, 125.9, 125.8, 125.7, 124.0, 120.9, 120.9, 118.5, 115.7, 115.7, 115.4, 115.3, 111.3, 111.1, 109.4, 103.2, 65.3, 54.9, 37.5, 33.7, 32.6, 32.1, 26.7, 26.6; ¹⁹F NMR: (282 MHz, CHLOROFORM-d) δ −113.53. Anal. (C₂₈H₂₈FN₃O₄) C, H, N.

### tert-Butyl (2S)-Cyclohexyl({[5-(2,6-dimethoxyphenyl)-1-(4-fluorophenyl)-1H-pyrazol-3-yl]carbonyl}amino)ethanoate (28a)

Following the general amide coupling procedure, 28a was obtained from 5-(2,6-dimethoxyphenyl)-1-(4-fluorophenyl)-1H-pyrazole-3-carboxylic acid (11i) and tert-butyl (2S)-amino-(cyclohexyl)ethanoate hydrochloride (12d) as an off-white powder in 94% yield. ¹H NMR (CDCl₃) δ 7.42-7.54 (m, 1H), 7.21-7.35 (m, 3H), 6.90-7.03 (m, 3H), 6.50 (dd, J=8.48, 13.85 Hz, 2H), 4.67 (dd, J=5.09, 9.14 Hz, 1H), 3.62 (s, 3H), 3.52 (s, 3H), 1.60-1.92 (m, 7H), 1.48-1.52 (m, 9H), 1.16-1.33 (m, 5H). MS (ESI) m/z: 536.5 (M+H⁺).

### (2S)-Cyclohexyl({[5-(2,6-dimethoxyphenyl)-1-(4-fluorophenyl)-1H-pyrazol-3-yl]carbonyl}amino)ethanoic Acid (28b)

Following the general tert-butyl ester hydrolysis procedure 28b was obtained as a white foam (83%) from 28a. ¹H NMR (CD₃OD) δ 7.23-7.42 (m, 3H), 6.99-7.15 (m, 2H), 6.76-6.86 (m, 1H), 6.62 (d, J=8.29 Hz, 2H), 4.57 (d, J=6.03 Hz, 1H), 3.60 (s, 6H), 1.74-2.10 (m, 7H), 1.21-1.44 (m, 4H). MS (ESI) m/z: 480.3 (M−H+).MS (ESI) m/z: 480.6 (M−H⁺). Anal. (C₂₆H₂₆FN₃O₅) C, H, N.

### Methyl 1-({[5-(2,6-dimethoxyphenyl)-1-(4-fluorophenyl)-1H-pyrazol-3-yl]carbonyl}amino)cyclo-hexanecarboxylate (29a)

Following the general amide coupling procedure 29a was prepared from 5-(2,6-dimethoxyphenyl)-1-(4-fluorophenyl)-1H-pyrazole-3-carboxylic acid (11i) and ethyl 1-aminocyclohexane-carboxylate hydrochloride (12b) as a colorless solid in 50% yield. ¹H NMR (CDCl₃) δ 7.33-7.25 (m, 4H), 7.00-6.90 (m, 2H), 6.50 (d, 8.5 Hz, 2H), 3.75 (s, 3H), 3.58 (s, 6H), 2.25-2.13 (m, 2H), 2.01-1.89 (m, 2H), 1.74-1.52 (m, 6H).

### 1-({[5-(2,6-Dimethoxyphenyl)-1-(4-fluorophenyl)-1H-pyrazol-3-yl]carbonyl}amino)cyclohexane-carboxylic Acid (29b)

Following the general methyl ester hydrolysis procedure, 29b was obtained from 29a as a white solid (89%). ¹H NMR (CDCl₃) δ 7.36-7.21 (m, 4H), 7.03-6.95 (m, 2H), 6.50 (d, J=8.5 Hz, 2H), 3.6 (s, 6H), 2.34-2.2 (m, 2H), 2.10-1.96 (m, 2H), 1.79-1.47 (m, 6H). MS (ESI) m/z: 510.5 (M−H⁺). Anal. (C₂₅H₂₆FN₃O₅) C, H, N.

### Methyl 1-(1-(4-Fluorophenyl)-5-(2-methoxyphenyl)-1H-pyrazole-3-carboxamido)cyclohexane-carboxylate (7a)

Following the general amide coupling procedure, 7a was prepared from 1-(4-fluorophenyl)-5-(2-methoxyphenyl)-1H-pyrazole-3-carboxylic acid (11j) and ethyl 1-aminocyclo-hexanecarboxylate hydrochloride (12b) as a solid (55% yield). ¹H NMR (CDCl₃) δ 1.46-1.56 (m, 6H), 1.88-2.02 (m, 2H), 2.15-2.25 (m, 2H), 3.48 (s, 3H), 3.75 (s, 3H), 6.80 (d, J=8.5 Hz, 1H), 6.94-7.04 (m, 3H), 7.18 (s, 1H), 7.23-7.31 (m, 3H), 7.36 (t, J=7.8 Hz, 1H).

### 1-({[1-(4-Fluorophenyl)-5-(2-methoxyphenyl)-1H-pyrazol-3-yl]carbonyl}amino)cyclo-hexane carboxylic Acid (7b)

Following the general methyl ester hydrolysis procedure, 7b was obtained from 7a as a white solid (85%). ¹H NMR (CDCl₃) δ 7.42-7.34 (m, 1H), 7.30-7.19 (m, 4H), 7.05-6.96 (m, 4H), 6.8 (d, 8.5 Hz, 1H), 3.43 (s, 3H), 2.3-2.2 (m, 2H), 2.10-1.96 (m, 2H), 1.79-1.47 (m, 6H). MS (ESI) m/z: 436.7 (M−H⁺). Anal. (C₂₄H₂₄FN₃O₄) C, H, N.

Pharmalogical Methods.

Calcium Mobilization Assay for NTS1 Receptor.

CHO-kl-rNTS1 cells were maintained in DMEM/F12 medium supplemented with 10% fetal bovine serum (Gibco), 100 U/ml penicillin/100 ug/ml streptomycin, 100 ug/ml normocin (Invivogen), and 250 ug/ml geneticin. For calcium mobilization assays, cells were plated at 30,000 cells/well in black, clear-bottom 96-well plates 24 hrs before the assay and incubated at 37° C., 5% CO₂. Prior to the assay, Calcium 5 dye (Molecular Devices) was reconstituted according to manufacturer instructions. The reconstituted dye was diluted 1:40 in assay buffer (lx HBSS, 20 mM HEPES, and 2.5 mM Probenicid (Sigma), pH 7.4). Growth media was removed and 200 ul of this diluted dye was added to each well. Plates were incubated for 45 min at 37° C., 5% CO₂ after dye addition. For agonist assays, cells were pre-treated with 1:10 addition of 10% DMSO in assay buffer and the plates were returned to 37° C., 5% CO₂ for 15 minutes. Full-log serial dilutions of the test compounds were made at 10× the desired final concentration in 1% DMSO assay buffer and warmed to 37° C. After the pre-treatment incubation, fluorescence intensity was measured on a FlexStation II fluorometric imaging plate reader (Molecular Devices). Relative fluorescence units (RFU) were measured before (20 readings) and after (40 readings) the agonist compound addition for a total 60 second read time (Excitation=485 nm, Emission=525 nm, cutoff=515 nm). For antagonist assays, cells were treated with Calcium dye for 45 min at 37° C., 5% CO₂ as with the agonist assays. Then the test antagonist compounds were added in 1% DMSO in assay buffer and the plate was incubated for 15 min at 37° C., 5% CO₂. Kₑ assays were run against dose-response curves of the control agonist NT and IC₅₀ assays were run against 1 nM NT.

Calcium Mobilization Assay for NTS2 Receptor.

CHO-kl-rNTS2 cells were maintained in DMEM/F12 supplemented with 10% FBS, pen/strep, 100 μg/ml normocin, and 400 μg/ml geneticin. For calcium mobilization assays, cells were plated at 25,000 cells/well in black, clear-bottom 96-well plates 24 hrs before the assay and incubated at 37° C., 5% CO₂. 100 μl reconstituted Calcium 5 dye (Molecular Devices, diluted 1:20 in assay buffer (1×HBSS, 20 mM HEPES, 2.5 mM Probenicid, pH 7.4)) was added per well and plates were incubated for 45 min at 37° C., 5% CO₂. For agonist assays, cells were pre-treated with 1:10 addition of 10% DMSO and the plates were returned to 37° C., 5% CO₂. Full-log serial dilutions of the test compounds were made at 10× the desired final concentration in 1% DMSO assay buffer and warmed to 37° C. After the pre-treatment incubation, fluorescence intensity was measured on a FLIPR Tetra fluorometric imaging plate reader (Molecular Devices). Relative Fluorescence Units (RFUs) were measured every second for 100 seconds (14 readings before compound addition to establish baseline fluorescence, 85 after) Exposure time 0.53 s, Gain=2000, Excitation intensity=30%, Gate open=10%. For antagonist assays, 1/10 volume of 10×concentration of the test compound dilutions in 10% DMSO in assay buffer was added to cells in lieu of the 10% DMSO only in assay buffer for the pre-treatment. Kₑ assays were run against dose-response curves of the control agonist 5b and IC₅₀ assays were run against the EC₈₀ of 5b (73 nM final concentration).

Competitive Binding Assays.

Relative binding affinity was evaluated using ¹²⁵I labeled neurotensin and CHO-kl cell lines over-expressing either the rNTS1 or rNTS2 receptor essentially as described by Gendron. Briefly, cells were plated at 100,000 cells/well in 24-well plates in complete DMEM/F12 medium supplemented with 10% fetal bovine serum, 100 units of penicillin/mL, 100 μg of streptomycin/mL, 0.1 mg/mL of normocin, and 250 μg/mL geneticin. Cells were incubated at 37° C. with 5% CO2 and 95% humidity for 48 hrs. Cells were then equilibrated for 10 minutes in Earle's buffer (130 mM NaCl, 5 mM KCl, 1.8 mM CaCl₂, 0.8 mM MgCl₂, and 20 mM HEPES, pH 7.4) supplemented with 0.1% BSA and 0.1% glucose, and then incubated with or without 10 nM test compound in 0.1 nM ¹²⁵I-NT at 37° C. for 30 min. Cells were washed twice in Earle's buffer, extracted in 1 ml 0.1N NaOH and counted in a Packard Cobra II Gamma Counter for 1 minute. Total binding was determined in the absence of test compound and non-specific binding was determined in the presence of 1.0 μM non-radiolabeled neurotensin.

Data Analysis.

To determine EC₅₀, IC₅₀, and Kₑ values, data were fit to a three-parameter logistic equation using GraphPad Prism software. Kᵢ values for radioligand binding assays were determined from ICso values using the equation of Cheng and Prusoff. All data are from at least three independent experiments run in duplicate wells.

### TABLES

TABLE 1
Data for Standards and Reference Compounds at the NTS1 and NTS2 Receptors

| | FLIPR Assays | | | | | | ¹²⁵I-NT |
|---|---|---|---|---|---|---|---|
| | | | | NTS2 | | | Binding |
| | NTS1 | | | | Eₘₐₓ | | NTS2 |
| | EC₅₀ | Eₘₐₓ | Kₑ | EC₅₀ | % of 5b ± | IC₅₀ | Kᵢ |
| | nM ± SEM | % NT ± SEM | nM ± SEM | nM ± SEM | SEM | nM ± SEM | nM ± SEM |
| NT | 0.04 ± 0.012 | 100 ± 3 | | NA* | | 18.5 ± 1.2 | 18.9 ± 3 |
| 1 | 0.01 ± 0.002 | 114 ± 7 | | NA | | 5.4 ± 0.6 | 33 ± 11 |
| 5a | | | 4.7 ± 0.8 | 120 ± 20 | 100 ± 3 | | 62 ± 35 |
| 5b | | | 1.5 ± 0.6 | 20 ± 5 | 100 ± 5 | | 6 ± 2 |
| 6 | NA* | | NA | 28 ± 4 | 16 ± 3 | | 33 ± 5 |
| 13 | NA | | NA | NA | | NA | >11 μM |

TABLE 2
Antagonism of NT induced calcium release at NTS1 compared to target compound induced
calcium release and binding affinity at NTS2 for 7-chloroquinolyl-substituted pyrazole carboxamides

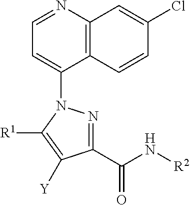

| | | | | FLIPR Assays | | | ¹²⁵\|-NT |
|---|---|---|---|---|---|---|---|
| | | | | | NTS2 | | Binding |
| | | | | NTS1 | | Eₘₐₓ | NTS2 |
| | | | | Kₑ | EC₅₀ | % 5b ± | Kᵢ |
| | R¹ | R² | Y | nM ± SEM | nM ± SEM | SEM | nM ± SEM |
| 5a | 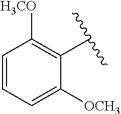 [SMILES: *c1c(OC)cccc1OC] | 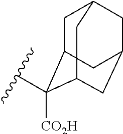 [SMILES: *C1(C(=O)O)C2CC3CC(C2)CC1C3] | H | 4.7 ± 0.8 | 120 ± 20 | 100 ± 15 | 62 ± 35 |
| 14b | 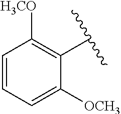 [SMILES: *c1c(OC)cccc1OC] | 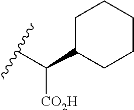 [SMILES: *[C@H](C(=O)O)C1CCCCC1] | H | 23 ± 6 | 217 ± 19 | 86 ± 3 | 644 ± 90 |
| 15b | 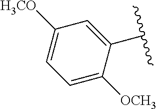 [SMILES: *c1cc(OC)ccc1OC] | 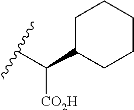 [SMILES: *[C@H](C(=O)O)C1CCCCC1] | H | 1275 ± 544 | 216 ± 29 | 19 ± 3 | 604 ± 141 |
| 16b | 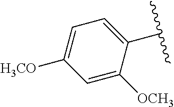 [SMILES: *c1ccc(OC)cc1OC] | 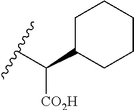 [SMILES: *[C@H](C(=O)O)C1CCCCC1] | H | >10 μM | 382 ± 33 | 12 ± 3 | 1585 ± 505 |
| 17b | 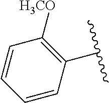 [SMILES: *c1ccccc1OC] | 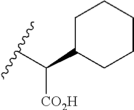 [SMILES: *[C@H](C(=O)O)C1CCCCC1] | H | 1682 ± 527 | 258 ± 14 | 25 ± 2 | 1418 ± 468 |
| 18b | 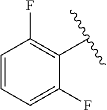 [SMILES: *c1c(F)cccc1F] | 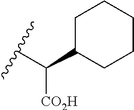 [SMILES: *[C@H](C(=O)O)C1CCCCC1] | H | >10 μM | 166 ± 3 | 15 ± 3 | 1001 ± 227 |
| 19b | 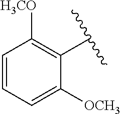 [SMILES: *c1c(OC)cccc1OC] | 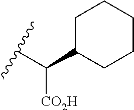 [SMILES: *[C@H](C(=O)O)C1CCCCC1] | Et | >10 μM | 94 ± 17 | 15 ± 1 | 1177 ± 155 |
| 20b | 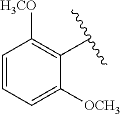 [SMILES: *c1c(OC)cccc1OC] | 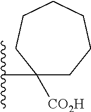 [SMILES: *C1(C(=O)O)CCCCCC1] | H | 42 ± 6 | 21 ± 5 | 89 ± 5 | 170 ± 71 |
| 21b | 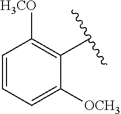 [SMILES: *c1c(OC)cccc1OC] | 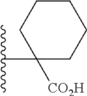 [SMILES: *C1(C(=O)O)CCCCC1] | H | 157 ± 45 | 29 ± 2 | 78 ± 7 | 151 ± 67 |
| 22b | 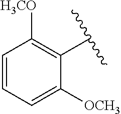 [SMILES: *c1c(OC)cccc1OC] | 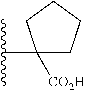 [SMILES: *C1(C(=O)O)CCCC1] | H | 222 ± 14 | 20 ± 4 | 76 ± 4 | 102 ± 27 |
| 23b | 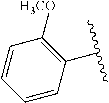 [SMILES: *c1ccccc1OC] | 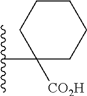 [SMILES: *C1(C(=O)O)CCCCC1] | H | 4549 ± 636 | 62 ± 9 | 35 ± 1 | 533 ± 153 |

TABLE 3
Antagonism of NT induced calcium release at NTS1 compared to test compound induced
calcium release and binding affinity at NTS2 for napthyl (A) and 4-F-phenyl (B) substituted
pyrazole carboxamides
A

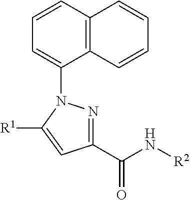

B

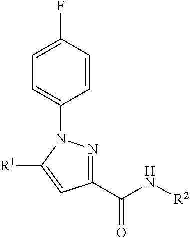

| | | | | FLIPR Assays | | | ¹²⁵\|-NT |
|---|---|---|---|---|---|---|---|
| | | | | | NTS2 | | Binding |
| | | | | NTS1 | | Eₘₐₓ | NTS2 |
| | | | | Kₑ | EC₅₀ | % of 5b | Kᵢ |
| # | | R¹ | R² | nM ± SEM | nM ± SEM | nM ± SE | nM ± SEM |
| 24b | A | 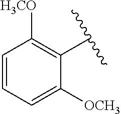 [SMILES: *c1c(OC)cccc1OC] | 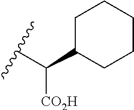 [SMILES: *[C@H](C(=O)O)C1CCCCC1] | 58 ± 6 | 159 ± 9 | 45 ± 1 | 536 ± 20 |
| 25b | A | 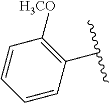 [SMILES: *c1ccccc1OC] | 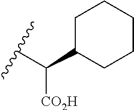 [SMILES: *[C@H](C(=O)O)C1CCCCC1] | 1404 ± 330 | 229 ± 8 | 18 ± 2 | 2687 ± 639 |
| 26b | A | 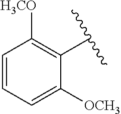 [SMILES: *c1c(OC)cccc1OC] | 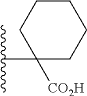 [SMILES: *C1(C(=O)O)CCCCC1] | 230 ± 79 | 18 ± 1.6 | 35 ± 1 | 116 ± 39 |
| 27b | A | 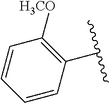 [SMILES: *c1ccccc1OC] | 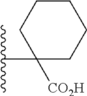 [SMILES: *C1(C(=O)O)CCCCC1] | 7380 ± 1130 | 108 ± 8 | 18 ± 2 | 694 ± 178 |
| 30 | B | 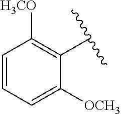 [SMILES: *c1c(OC)cccc1OC] | 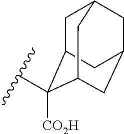 [SMILES: *C1(C(=O)O)C2CC3CC(C2)CC1C3] | 191 ± 47 | 68 ± 25 | 34 ± 5 | 110 ± 29 |
| 31 | B | 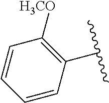 [SMILES: *c1ccccc1OC] | 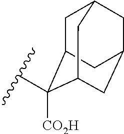 [SMILES: *C1(C(=O)O)C2CC3CC(C2)CC1C3] | 3814 ± 422 | 138 ± 55 | 42 ± 12 | 23 ± 5 |
| 28b | B | 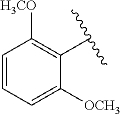 [SMILES: *c1c(OC)cccc1OC] | 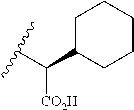 [SMILES: *[C@H](C(=O)O)C1CCCCC1] | 3344 ± 549 | 271 ± 24 | 22 ± 5 | 622 ± 245 |
| 29b | B | 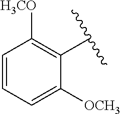 [SMILES: *c1c(OC)cccc1OC] | 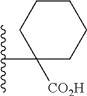 [SMILES: *C1(C(=O)O)CCCCC1] | >10 μM | 19 ± 3 | 12 ± 0.5 | 140 ± 29 |
| 7b | B | 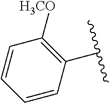 [SMILES: *c1ccccc1OC] | 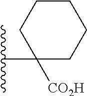 [SMILES: *C1(C(=O)O)CCCCC1] | >10 μM | 12 ± 6 | 7 ± 2 | 153 ± 10 |

TABLE 4
Selectivity Ratios for 29b and 7b at the NTS1 and NTS2 Receptors
Determined Using ¹²⁵I-NT Radioligand Binding

| | NTS1 Kᵢ | NTS2 Kᵢ | |
|---|---|---|---|
| # | nM ± SEM | nM ± SEM | NTS2/NTS1 |
| 29b | 3210 ± 879 | 140 ± 29 | 23 |
| 7b | >25 uM | 153 ± 10 | 161 |
| 31 | 4327 ± 656 | 23 ± 5 | 188 |

TABLE 5
Elemental Analysis

| | | Calculated | | | Found | | |
|---|---|---|---|---|---|---|---|
| Compd | Formula | C | H | N | C | H | N |
| 7b | C₂₄H₂₄FN₃O₄ | 65.89 | 5.53 | 9.61 | 65.67 | 5.62 | 9.90 |
| 13 | C₃₂H₃₃N₃O₃•Et₂O | 74.33 | 7.45 | 7.22 | 74.61 | 7.26 | 6.93 |
| 14b | C₂₉H₂₉ClN₄O₅•H₂O | 61.43 | 5.51 | 9.88 | 61.73 | 5.42 | 9.83 |
| 15b | C₂₉H₂₉ClN₄O₅•H₂O | 61.43 | 5.51 | 9.88 | 61.71 | 5.74 | 9.51 |
| 16b | C₂₉H₂₉ClN₄O₅•Et₂O | 63.61 | 6.31 | 8.99 | 63.87 | 6.22 | 9.31 |
| 17b | C₂₈H₂₇ClN₄O₄ | 64.80 | 5.24 | 10.80 | 64.94 | 5.55 | 11.17 |
| 18b | C₂₇H₂₃ClF₂N₄O₃ | 61.78 | 4.42 | 10.67 | 61.85 | 4.68 | 10.33 |
| 19b | C₃₁H₃₃ClN₄O₆•1.5H₂O | 60.93 | 5.77 | 9.17 | 60.62 | 6.01 | 9.12 |
| 20b | C₂₉H₂₉ClN₄O₅ | 63.44 | 5.32 | 10.20 | 63.52 | 5.39 | 10.15 |
| 21b | C₂₈H₂₇ClN₄O₅•H₂O | 60.81 | 5.29 | 10.13 | 60.52 | 5.36 | 9.94 |
| 22b | C₂₇H₂₅ClN₄O₅ | 62.25 | 4.84 | 10.75 | 61.89 | 4.98 | 10.73 |
| 23b | C₂₇H₂₅ClN₄O₄ | 64.22 | 4.99 | 11.10 | 64.44 | 5.03 | 10.81 |
| 24b | C₃₀H₃₁N₃O₅ | 70.16 | 6.08 | 8.18 | 70.09 | 6.36 | 7.94 |
| 25b | C₂₉H₂₉N₃O₄ | 72.03 | 6.04 | 8.69 | 71.74 | 6.22 | 8.67 |
| 26b | C₂₉H₂₉N₃O₅•H₂O | 67.30 | 6.04 | 8.12 | 67.43 | 6.27 | 8.41 |
| 27b | C₂₈H₂₇N₃O₄ | 71.62 | 5.80 | 8.95 | 71.38 | 6.02 | 8.59 |
| 28b | C₂₆H₂₈FN₃O₅ | 64.85 | 5.86 | 8.73 | 64.64 | 5.79 | 8.68 |
| 29b | C₂₅H₂₆FN₃O₅•Et₂O | 64.31 | 6.70 | 7.76 | 64.13 | 6.76 | 8.02 |
| 30 | C₂₉H₃₀FN₃O₅ | 67.04 | 5.82 | 8.09 | 67.35 | 5.87 | 7.85 |
| 31 | C₂₈H₂₈FN₃O₄ | 68.70 | 5.77 | 8.58 | 67.88 | 5.60 | 8.73 |

### 7. REFERENCES

1. Caraway, R.; Leeman, S. E. The isolation of a new hypotensive peptide, neurotensin, from bovine hypothalami. J. Biol. Chem 1973, 248, 6854-6861.
2. Hokfelt, T.; Everitt, B. J.; Theodorsson-Norheim, E.; Goldstein, M. Occurrence of neurotensinlike immunoreactivity in subpopulations of hypothalamic, mesencephalic, and medullary catecholamine neurons. J Comp Neurol 1984, 222, 543-59.
3. Kalivas, P. W.; Miller, J. S. Neurotensin neurons in the ventral tegmental area project to the medial nucleus accumbens. Brain Res 1984, 300, 157-60.
4. Kalivas, P. W.; Nemeroff, C. B.; Prange, A. J., Jr. Neuroanatomical site specific modulation of spontaneous motor activity by neurotensin. Eur J Pharmacol 1982, 78, 471-4.
5. Ford, A. P.; Marsden, C. A. In vivo neurochemical and behavioural effects of intracerebrally administered neurotensin and D-Trp11-neurotensin on mesolimbic and nigrostriatal dopaminergic function in the rat. Brain Res 1990, 534, 243-50.
6. Nemeroff, C. B.; Hernandez, D. E.; Luttinger, D.; Kalivas, P. W.; Prange, A. J., Jr. Interactions of neurotensin with brain dopamine systems. Ann N Y Acad Sci 1982, 400, 330-44.
7. Clineschmidt, B. V.; McGuffin, J. C. Neurotensin administered intracisternally inhibits responsiveness of mice to noxious stimuli. Eur J Pharmacol 1977, 46, 395-6.
8. Nemeroff, C. B.; Osbahr, A. J., 3rd; Manberg, P. J.; Ervin, G. N.; Prange, A. J., Jr. Alterations in nociception and body temperature after intracisternal administration of neurotensin, beta-endorphin, other endogenous peptides, and morphine. Proc Natl Acad Sci USA 1979, 76, 5368-71.
9. Tanaka, K.; Masu, M.; Nakanishi, S. Structure and functional expression of the cloned rat neurotensin receptor. Neuron 1990, 4, 847-854.
10. Vita, N.; Laurent, P.; Lefort, S.; Chalon, P.; Dumont, X.; Kaghad, M.; Gully, D.; Le Fur, G.; Ferrara, P.; Caput, D. Cloning and expression of a complementary DNA encoding a high affinity human neurotensin receptor. FEBS Lett 1993, 317, 139-42.
11. Chalon, P.; Vita, N.; Kaghad, M.; Guillemot, M.; Bonnin, J.; Delpech, B.; Le Fur, G.; Ferrara, P.; Caput, D. Molecular cloning of a levocabastine-sensitive neurotensin binding site. FEBS Lett 1996, 386, 91-4.
12. Mazella, J.; Botto, J.; Guillemare, E.; Coppola, T.; Sarret, P.; Vincent, J. P. Structure, Functional Expression, and Cerebral Localization of the Levocabastine-Sensitive Neurotensin/Neuromedin N Receptor from Mouse Brain. Journal of Neuroscience 1996, 16, 5613-5620.
13. Mazella, J.; Zsurger, N.; Navarro, V.; Chabry, J.; Kaghad, M.; Caput, D.; Ferrara, P.; Vita, N.; Gully, D.; Maffrand, J. P.; Vincent, J. P. The 100-kDa neurotensin receptor is gp95/sortilin, a non-G-protein-coupled receptor. J Biol Chem 1998, 273, 26273-6.
14. Nemeroff, C. B. Neurotensin: perchance an endogenous neuroleptic? Biol Psychiatry 1980, 15, 283-302.
15. Kitabgi, P. Targeting neurotensin receptors with agonists and antagonists for therapeutic purposes. Curr Opin Drug Discov Devel 2002, 5, 764-76.
16. Boules, M.; Fredrickson, P.; Richelson, E. Neurotensin agonists as an alternative to antipsychotics. Expert Opin Investig Drugs 2005, 14, 359-69.
17. Tatetsu, S.; Takaki, M.; Miyagawa, T.; Kozuma, Y.; Kozuma, Y. [A study on the experimental production in the brain of histopathological changes resembling schizophrenia by chronic methamphetamine administration]. Seishin Shinkeigaku Zasshi 1967, 69, 1363-70.
18. Machiyama, Y. Chronic methamphetamine intoxication model of schizophrenia in animals. Schizophr Bull 1992, 18, 107-13.
19. Frankel, P. S.; Hoonakker, A. J.; Alburges, M. E.; McDougall, J. W.; McFadden, L. M.; Fleckenstein, A. E.; Hanson, G. R. Effect of methamphetamine self-administration on neurotensin systems of the basal ganglia. J Pharmacol Exp Ther 2011, 336, 809-15.
20. Hanson, G. R.; Hoonakker, A. J.; Robson, C. M.; McFadden, L. M.; Frankel, P. S.; Alburges, M. E. Response of Neurotensin Basal Ganglia Systems during Extinction of Methamphetamine Self-Administration in Rat. J Pharmacol Exp Ther 2013, 346, 173-81.
21. Dobner, P. R. Neurotensin and pain modulation. Peptides 2006, 27, 2405-14.
22. Boules, M.; Johnston, H.; Tozy, J.; Smith, K.; Li, Z.; Richelson, E. Analgesic synergy of neurotensin receptor subtype 2 agonist NT79 and morphine. Behav Pharmacol 2011, 22, 573-81.
23. Bredeloux, P.; Cavelier, F.; Dubuc, I.; Vivet, B.; Costentin, J.; Martinez, J. Synthesis and biological effects of c(Lys-Lys-Pro-Tyr-Ile-Leu-Lys-Lys-Pro-Tyr-Ile-Leu) (JMV2012), a new analogue of neurotensin that crosses the blood-brain barrier. J Med Chem 2008, 51, 1610-6.
24. Bredeloux, P.; Costentin, J.; Dubuc, I. Interactions between NTS2 neurotensin and opioid receptors on two nociceptive responses assessed on the hot plate test in mice. Behav Brain Res 2006.
25. Smith, K. E.; Boules, M.; Williams, K.; Richelson, E. NTS1 and NTS2 mediate analgesia following neurotensin analog treatment in a mouse model for visceral pain. Behav Brain Res 2012, 232, 93-7.
26. Hughes, F. M.; Shaner, B. E.; May, L. A.; Zotian, L.; Brower, J. O.; Woods, R. J.; Cash, M.; Morrow, D.; Massa, F.; Mazella, J.; Dix, T. A. Identification and Functional Characterization of a Stable, Centrally Active Derivative of the Neurotensin (8-13) Fragment as a Potential First-in-Class Analgesic. J Med Chem 2010.
27. Sarret, P.; Esdaile, M. J.; Perron, A.; Martinez, J.; Stroh, T.; Beaudet, A. Potent spinal analgesia elicited through stimulation of NTS2 neurotensin receptors. J Neurosci 2005, 25, 8188-96.
28. Tetreault, P.; Beaudet, N.; Perron, A.; Belleville, K.; Rene, A.; Cavelier, F.; Martinez, J.; Stroh, T.; Jacobi, A. M.; Rose, S. D.; Behlke, M. A.; Sarret, P. Spinal NTS2 receptor activation reverses signs of neuropathic pain. FASEB J 2013.
29. Finnerup, N. B.; Sindrup, S. H.; Jensen, T. S. The evidence for pharmacological treatment of neuropathic pain. Pain 2010, 150, 573-81.
30. Carraway, R. L., S. E. In Structural requirements for the biological activity of neurotensin, a new vasoactive peptide, Peptides: Chemistry, Structure and Biology, 1975; Walter, R., Meienhofer, J., Ed. Ann Arbor Science, Ann Arbor, Mich.: 1975; pp 679-685.
31. Dubuc, I.; Sarret, P.; Labbe-Jullie, C.; Botto, J. M.; Honore, E.; Bourdel, E.; Martinez, J.; Costentin, J.; Vincent, J. P.; Kitabgi, P.; Mazella, J. Identification of the receptor subtype involved in the analgesic effect of neurotensin. J Neurosci 1999, 19, 503-10.
32. Doulut, S.; Rodriguez, M.; Lugrin, D.; Vecchini, F.; Kitabgi, P.; Aumelas, A.; Martinez, J. Reduced peptide bond pseudopeptide analogues of neurotensin. Pept Res 1992, 5, 30-8.
33. Boules, M.; Liang, Y.; Briody, S.; Miura, T.; Fauq, I.; Oliveros, A.; Wilson, M.; Khaniyev, S.; Williams, K.; Li, Z.; Qi, Y.; Katovich, M.; Richelson, E. NT79: A novel neurotensin analog with selective behavioral effects. Brain Res 2010, 1308, 35-46.
34. Einsiedel, J.; Held, C.; Hervet, M.; Plomer, M.; Tschammer, N.; Hubner, H.; Gmeiner, P. Discovery of highly potent and neurotensin receptor 2 selective neurotensin mimetics. J Med Chem 2011, 54, 2915-23.
35. Held, C.; Plomer, M.; Hubner, H.; Meltretter, J.; Pischetsrieder, M.; Gmeiner, P. Development of a Metabolically Stable Neurotensin Receptor 2 (NTS2) Ligand. Chem Med Chem 2012.
36. Gully, D.; Canton, M.; Boigegrain, R.; Jeanjean, F.; Molimard, J. C.; Poncelet, M.; Gueudet, C.; Heaulme, M.; Leyris, R.; Brouard, A.; et al. Biochemical and pharmacological profile of a potent and selective nonpeptide antagonist of the neurotensin receptor. Proc Natl Acad Sci USA 1993, 90, 65-9.
37. Gully, D.; Labeeuw, B.; Boigegrain, R.; Oury-Donat, F.; Bachy, A.; Poncelet, M.; Steinberg, R.; Suaud-Chagny, M. F.; Santucci, V.; Vita, N.; Pecceu, F.; Labbe-Jullie, C.; Kitabgi, P.; Soubrie, P.; Le Fur, G.; Maffrand, J. P. Biochemical and pharmacological activities of SR 142948A, a new potent neurotensin receptor antagonist. J Pharmacol Exp Ther 1997, 280, 802-12.
38. Dahl, R.; Pedersen, B.; Larsen, B. Intranasal levocabastine for the treatment of seasonal allergic rhinitis: a multicentre, double-blind, placebo-controlled trial. Rhinology 1995, 33, 121-5.
39. Yamada, M.; Yamada, M.; Lombet, A.; Forgez, P.; Rostene, W. Distinct functional characteristics of levocabastine sensitive rat neurotensin NT2 receptor expressed in Chinese hamster ovary cells. Life Sci 1998, 62, PL 375-80.
40. Mazella, J.; Botto, J. M.; Guillemare, E.; Coppola, T.; Sarret, P.; Vincent, J. P. Structure, functional expression, and cerebral localization of the levocabastine-sensitive neurotensin/neuromedin N receptor from mouse brain. J Neurosci 1996, 16, 5613-20.
41. Richard, F.; Barroso, S.; Martinez, J.; Labbe-Jullie, C.; Kitabgi, P. Agonism, inverse agonism, and neutral antagonism at the constitutively active human neurotensin receptor 2. Mol Pharmacol 2001, 60, 1392-8.
42. Vita, N.; Oury-Donat, F.; Chalon, P.; Guillemot, M.; Kaghad, M.; Bachy, A.; Thurneyssen, O.; Garcia, S.; Poinot-Chazel, C.; Casellas, P.; Keane, P.; Le Fur, G.; Maffrand, J. P.; Soubrie, P.; Caput, D.; Ferrara, P. Neurotensin is an antagonist of the human neurotensin NT2 receptor expressed in Chinese hamster ovary cells. Eur J Pharmacol 1998, 360, 265-72.
43. Labeeuw, B.; Gully, D.; Jeanjean, F.; Molimard, J.; Boigegrain, R. Substituted 1-Phenyl-3-pyrazolecarboxamides Active on the Neurotensin Receptors, Their Preparation and Pharmaceutical Compositions Containing Them. 5,723,483, 1998.
44. Jiang, J.; Huang, W.; Zhai, J.; Liu, H.; Cai, Q.; Xu, L.; Wang, W.; Ji, Y. ‘One-pot’ synthesis of 4-substituted 1,5-diaryl-1H-pyrazole-3-carboxylates via lithium tert-butoxide-mediated sterically hindered Claisen condensation and Knorr reaction. Tetrahedron 2013, 69, 627-635.
45. Baxendale, I. R.; Cheung, S.; Kitching, M. O.; Ley, S. V.; Shearman, J. W. The synthesis of neurotensin antagonist SR 48692 for prostate cancer research. Biorg. Med. Chem. 2013, 21, 4378-4387.
46. Lindh, J.; Sojberg, P. J. R.; M., L. Synthesis of aryl ketones by palladium(II)-catalyzed decarboxylative addition of benzoic acids to nitriles. Angewandte Chemie Int. Ed. 2010, 49, 7733-7737.
47. Quéré, L. Etude de l′interaction ligand-récepteur neurotensinergique. PhD Thesis 1995.
48. Nagasawa, H. T.; Eberling, J. A.; Shirota, F. N. 2-Aminoadamantane-2-carboxylic Acid, a Rigid, Achiral, Tricyclic alpha-Amino Acid with Transport Inhibitory Properties. J. Med. Chem 1973, 16, 823-826.
49. Gendron, L.; Perron, A.; Payet, M. D.; Gallo-Payet, N.; Sarret, P.; Beaudet, A. Low-affinity neurotensin receptor (NTS2) signaling: internalization-dependent activation of extracellular signal-regulated kinases 1/2. Mol Pharmacol 2004, 66, 1421-30.
50. Gaddum, J. H. Theories of drug antagonism. Pharmacol Rev 1957, 9, 211-218.
51. Kenakin, T.; Jenkinson, S.; Watson, C. Determining the potency and molecular mechanism of action of insurmountable antagonists. J Pharmacol Exp Ther 2006, 319, 710-23.
52. Labbe-Jullie, C.; Barroso, S.; Nicolas-Eteve, D.; Reversat, J. L.; Botto, J. M.; Mazella, J.; Bernassau, J. M.; Kitabgi, P. Mutagenesis and modeling of the neurotensin receptor NTR1. Identification of residues that are critical for binding SR 48692, a nonpeptide neurotensin antagonist. J Biol Chem 1998, 273, 16351-7.
53. Labbe-Jullie, C.; Botto, J. M.; Mas, M. V.; Chabry, J.; Mazella, J.; Vincent, J. P.; Gully, D.; Maffrand, J. P.; Kitabgi, P. [3H]SR 48692, the first nonpeptide neurotensin antagonist radioligand: characterization of binding properties and evidence for distinct agonist and antagonist binding domains on the rat neurotensin receptor. Mol Pharmacol 1995, 47, 1050-6.
54. Henry, J. A.; Horwell, D. C.; Meecham, K. G.; Rees, D. C. A structure-affinity study of the amino acid side-chains in neurotensin: N and C terminal deletions and Ala-scan. Bioorg Med Chem. Lett. 1993, 3, 949-952.
55. Roussy, G.; Dansereau, M. A.; Baudisson, S.; Ezzoubaa, F.; Belleville, K.; Beaudet, N.; Martinez, J.; Richelson, E.; Sarret, P. Evidence for a role of NTS2 receptors in the modulation of tonic pain sensitivity. Mol Pain 2009, 5, 38.
56. Smith, D. J.; Hawranko, A. A.; Monroe, P. J.; Gully, D.; Urban, M. O.; Craig, C. R.; Smith, J. P.; Smith, D. L. Dose-dependent pain-facilitatory and -inhibitory actions of neurotensin are revealed by SR 48692, a nonpeptide neurotensin antagonist: influence on the antinociceptive effect of morphine. J Pharmacol Exp Ther 1997, 282, 899-908.
57. Costa, F. G.; Frussa-Filho, R.; Felicio, L. F. The neurotensin receptor antagonist, SR48692, attenuates the expression of amphetamine-induced behavioural sensitisation in mice. Eur J Pharmacol 2001, 428, 97-103.
58. Gully, D.; Lespy, L.; Canton, M.; Rostene, W.; Kitabgi, P.; Le Fur, G.; Maffrand, J. P. Effect of the neurotensin receptor antagonist SR48692 on rat blood pressure modulation by neurotensin. Life Sci 1996, 58, 665-74.
59. Quéré, L.; Boigegrain, R.; Jeanjean, F.; Gully, D.; Evrard, G.; Durant, F. Structural requirements of non-peptide neurotensin receptor antagonists. J. Chem. Soc., Perkin Trans. 1996, 2, 2639-2646.
60. Thomas, J. B.; Navarro, H.; Warner, K. R.; Gilmour, B. The identification of nonpeptide neurotensin receptor partial agonists from the potent antagonist SR48692 using a calcium mobilization assay. Bioorg Med Chem Lett 2009, 19, 1438-41.

It is to be understood that, while the invention has been described in conjunction with the detailed description, thereof, the foregoing description is intended to illustrate and not limit the scope of the invention. Other aspects, advantages, and modifications of the invention are within the scope of the claims set forth below. All publications, patents, and patent applications cited in this specification are herein incorporated by reference as if each individual publication or patent application were specifically and individually indicated to be incorporated by reference.

1. A compound represented by the Formula I: 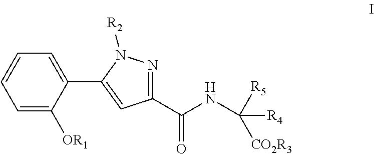
or a pharmaceutically acceptable salt, a prodrug, or a salt of a prodrug,
wherein
R₁ is adamantanyl, aryl, C₁₋₈ alkyl, C₁₋₈ alkyl(aryl), C₁₋₈ alkyl (C₃₋₈ cycloalkyl), C₂₋₈ alkenyl, C₃₋₈ alkynyl, C₃₋₈ cycloalkyl;
R₂ is aryl, C₁₋₈ alkyl(aryl);
R₃ is adamantanyl, aryl, C₁₋₈ alkyl, C₁₋₈ alkyl(aryl), C₁₋₈ alkyl (C₃₋₈ cycloalkyl), C₂₋₈ alkenyl, C₃₋₈ alkynyl, C₃₋₈ cycloalkyl or H; and
R₄ and R₅ are independently adamantanyl, aryl, C₁₋₈ alkyl, C₁₋₈ alkyl(aryl), C₁₋₈ alkyl (C₃₋₈ cycloalkyl), C₂₋₈ alkenyl, C₃₋₈ alkynyl, C₃₋₈ cycloalkyl, or H; or R₄ and R₅ together make a 4-8 member ring which may be substituted with one or more heteroatoms.

2. The compound of claim 1, wherein R₁ is C₁₋₈ alkyl.

3. The compound of claim 2, wherein R₁ is C₁₋₃ alkyl.

4. The compound of claim 1, wherein R₂ is aryl and the aryl moiety is substituted with a halogen.

5. The compound of claim 4, wherein R₂ is fluoroaryl.

6. The compound of claim 5, wherein R₂ is fluorophenyl.

7. The compound of claim 4, wherein R₂ is chloroaryl.

8. The compound of claim 7, wherein R₂ is chloroquinolinyl.

9. The compound of claim 1, wherein R₂ is an unsubstituted aryl.

10. The compound of claim 9, wherein the unsubstituted aryl moiety R₂ is napthyl.

11. The compound of claim 1, wherein R₄ and R₅ together make a 4-8 member ring.

12. The compound of claim 11, wherein R₄ and R₅ together make a C₅₋₈ cycloalkyl ring.

13. The compound of claim 12, wherein R₁ is C₁₋₃ alkyl and R₂ is fluoroaryl.

14. The compound of claim 1 having the structure of any of compounds 7b, 14b, 15b, 16b, 17b, 18b, 19b, 20b, 21b, 22b, 23b, 24b, 25b, 26b, 27b, 28b, 29b, 30 or 31 as set forth in Table 2 or 3.

15. A pharmaceutical composition comprising at least one pharmaceutically acceptable excipient and a therapeutically effective amount of the compound of claim 1.

16. The pharmaceutical composition of claim 15, wherein the compound is present in amount effective for the treatment of pain.

17. The pharmaceutical composition of claim 16, wherein the pain is chronic pain.

18. The pharmaceutical composition of claim 16, wherein the pain is neuropathic pain.

19. A method of treating a neurotensin 2 receptor (NTS2)-related disorder in a subject which comprises administering to the subject the compound of claim 1.

20. The method of claim 19, wherein the neurotensin 2 receptor (NTS2)-related disorder is pain.

21. The method of claim 20, wherein the pain is chronic pain.

22. The method of claim 20, wherein the pain is neuropathic pain.
